SEQUENCE LISTING (44 sequences)

SEQ ID NO: 1 - DNA, 27 bp, Artificial Sequence
  Synthetic Primer
tgggcggacctgattgttttcgccggc

SEQ ID NO: 2 - DNA, 30 bp, Artificial Sequence
  Synthetic Primer
gctcttaaggctggcaatctcggcttcgcc

SEQ ID NO: 3 - DNA, 30 bp, Artificial Sequence
  Synthetic Primer
tgcgatcgacgctggagaaggacaacaccg

SEQ ID NO: 4 - DNA, 30 bp, Artificial Sequence
  Synthetic Primer
tgtagtcggccgacacctcctcgatgacgc

SEQ ID NO: 5 - DNA, 50 bp, Artificial Sequence
  Synthetic Primer
ctggtgatcgcgtccttaccggttttttccggtaaggacgcgatcaccag

SEQ ID NO: 6 - DNA, 45 bp, Artificial Sequence
  Synthetic Primer
gtgatcgcgtccttaccgttttttcggtaaggacgcgatcaccag

SEQ ID NO: 7 - DNA, 46 bp, Artificial Sequence
  Synthetic Primer
tgatcgcgtccttaccggttttttccggtaaggacgcgatcaccag

SEQ ID NO: 8 - DNA, 46 bp, Artificial Sequence
  Synthetic Primer
tacggttccggcgttaccttttttggtaacgccggaaccgtaccag

SEQ ID NO: 9 - DNA, 47 bp, Artificial Sequence
  Synthetic Primer
gatcgcgtccttaccggtttttttaccggtaaggacgcgatcaccag

SEQ ID NO: 10 - DNA, 48 bp, Artificial Sequence
  Synthetic Primer
atcgcgtccttaccggttttttttaaccggtaaggacgcgatcaccag

SEQ ID NO: 11 - DNA, 49 bp, Artificial Sequence
  Synthetic Primer
tcgcgtccttaccggttcttttttgaaccggtaaggacgcgatcaccag

SEQ ID NO: 12 - DNA, 50 bp, Artificial Sequence
  Synthetic Primer
cgcgtccttaccggttccttttttggaaccggtaaggacgcgatcaccag

SEQ ID NO: 13 - DNA, 51 bp, Artificial Sequence
  Synthetic Primer
gcgtccttaccggttccgttttttcggaaccggtaaggacgcgatcaccag

SEQ ID NO: 14 - DNA, 22 bp, Artificial Sequence
  Synthetic Primer
ccggtaaggacgcgatcaccag

SEQ ID NO: 15 - DNA, 22 bp, Artificial Sequence
  Synthetic Primer
cagcagggctcttcgtcagctc

SEQ ID NO: 16 - DNA, 30 bp, Artificial Sequence
  Synthetic Primer
tgttgtcccatttcgtcggggtgttcgtcc

SEQ ID NO: 17 - DNA, 44 bp, Artificial Sequence
  Synthetic Primer
tggctcagctggctggtgcttttttgcaccagccagctgagcca

SEQ ID NO: 18 - DNA, 41 bp, Artificial Sequence
  Synthetic Primer
ctcagctggctggtgcttttttgcaccagccagctgagcca

SEQ ID NO: 19 - DNA, 42 bp, Artificial Sequence
  Synthetic Primer
tcagctggctggtgccttttttggcaccagccagctgagcca

SEQ ID NO: 20 - DNA, 42 bp, Artificial Sequence
  Synthetic Primer
cggtggccgctatcgtttttttacgatagcggccaccggcca

SEQ ID NO: 21 - DNA, 43 bp, Artificial Sequence
  Synthetic Primer
cagctggctggtgccgttttttcggcaccagccagctgagcca

SEQ ID NO: 22 - DNA, 44 bp, Artificial Sequence
  Synthetic Primer
agctggctggtgccgatttttttcggcaccagccagctgagcca

SEQ ID NO: 23 - DNA, 20 bp, Artificial Sequence
  Synthetic Primer
ggcaccagccagctgagcca

SEQ ID NO: 24 - DNA, 22 bp, Artificial Sequence
  Synthetic Primer
gcccggcacgctcacgtgacag

SEQ ID NO: 25 - DNA, 17 bp, Artificial Sequence
  Synthetic Primer
ccgactgttggcgctgg

SEQ ID NO: 26 - DNA, 50 bp, Artificial Sequence
  Synthetic Primer
ctggtgatcgcgtccttaccggttttttccggtaaggacgcgatcaccac

SEQ ID NO: 27 - DNA, 45 bp, Artificial Sequence
  Synthetic Primer
gtgatcgcgtccttaccgttttttcggtaaggacgcgatcaccac

SEQ ID NO: 28 - DNA, 46 bp, Artificial Sequence
  Synthetic Primer
tgatcgcgtccttaccggttttttccggtaaggacgcgatcaccac

SEQ ID NO: 29 - DNA, 46 bp, Artificial Sequence
  Synthetic Primer
tacggttccggcgttaccttttttggtaacgccggaaccgtaccac

SEQ ID NO: 30 - DNA, 47 bp, Artificial Sequence
  Synthetic Primer
gatcgcgtccttaccggtttttttaccggtaaggacgcgatcaccac

SEQ ID NO: 31 - DNA, 48 bp, Artificial Sequence
  Synthetic Primer
atcgcgtccttaccggttttttttaaccggtaaggacgcgatcaccac

SEQ ID NO: 32 - DNA, 49 bp, Artificial Sequence
  Synthetic Primer
tcgcgtccttaccggttcttttttgaaccggtaaggacgcgatcaccac

SEQ ID NO: 33 - DNA, 22 bp, Artificial Sequence
  Synthetic Primer
ccggtaaggacgcgatcaccac

SEQ ID NO: 34 - DNA, 22 bp, Artificial Sequence
  Synthetic Primer
cagcagggctcttcgtcagctc

SEQ ID NO: 35 - DNA, 30 bp, Artificial Sequence; identical to SEQ ID NO: 16
  Synthetic Primer

SEQ ID NO: 36 - DNA, 44 bp, Artificial Sequence
  Synthetic Primer
tggctcagctggctggtgcttttttgcaccagccagctgagcct

SEQ ID NO: 37 - DNA, 41 bp, Artificial Sequence
  Synthetic Primer
ctcagctggctggtgcttttttgcaccagccagctgagcct

SEQ ID NO: 38 - DNA, 42 bp, Artificial Sequence
  Synthetic Primer
tcagctggctggtgccttttttggcaccagccagctgagcct

SEQ ID NO: 39 - DNA, 42 bp, Artificial Sequence
  Synthetic Primer
cggtggccgctatcgtttttttacgatagcggccaccggcct

SEQ ID NO: 40 - DNA, 43 bp, Artificial Sequence
  Synthetic Primer
cagctggctggtgccgttttttcggcaccagccagctgagcct

SEQ ID NO: 41 - DNA, 44 bp, Artificial Sequence
  Synthetic Primer
agctggctggtgccgatttttttcggcaccagccagctgagcct

SEQ ID NO: 42 - DNA, 20 bp, Artificial Sequence
  Synthetic Primer
ggcaccagccagctgagcct

SEQ ID NO: 43 - DNA, 22 bp, Artificial Sequence
  Synthetic Primer
gcccggcacgctcacgtgacag

SEQ ID NO: 44 - DNA, 17 bp, Artificial Sequence
  Synthetic Primer
ccgactgttggcgctgg

(19) United States
(12) Patent Application Publication
(10) Pub. No.: US 2017/0051343 A1
(43) Pub. Date: Feb. 23, 2017

(54) STRAND EXCHANGE HAIRPIN PRIMERS THAT GIVE HIGH ALLELIC DISCRIMINATION

(71) Applicant: THE BOARD OF REGENTS OF THE UNIVERSITY OF TEXAS SYSTEM, AUSTIN, TX (US)

(72) Inventors: Michelle BYROM, Austin, TX (US); Sanchita BHADRA, Austin, TX (US); Yu Sherry JIANG, Austin, TX (US); Andrew Ellington, Austin, TX (US)

(21) Appl. No.: 15/118,571

(22) PCT Filed: Feb. 13, 2015
(86) PCT No.: PCT/US15/15936

§ 371 Date: Aug. 12, 2016

Related U.S. Application Data
(60) Provisional application No. 61/940,021, filed on Feb. 14, 2014.

(51) Int. Cl. C12Q 1/68

(52) U.S. Cl. CPC C12Q 1/6858

(57) ABSTRACT

Provided herein are compositions and methods for identification of the presence or absence of a particular sequence, such as a single nucleotide polymorphism. Employed herein are particular primers that comprise a hairpin and a single strand extension at the 3′ end, the single strand extension in which at least one nucleotide is mismatched compared to a target particular sequence. Strand displacement that leads to additional binding of the primer and extension of the primer occurs following initial binding of the primer to the nucleic acid comprising the particular sequence.

### STATEMENT REGARDING FEDERALLY SPONSORED RESEARCH OR DEVELOPMENT

This invention was made with government support under 5U54EB015403-02 awarded by the National Institutes of Health and HDTRA-1-13-1-0031 awarded by the Defense Advanced Research Projects Agency. The government has certain rights in the invention.

This application claims priority to U.S. Provisional Patent Application Ser. No. 61/940,021, filed Feb. 14, 2015, which is incorporated by reference herein in its entirety.

### TECHNICAL FIELD

The field of the disclosure includes at least molecular biology, cell biology, diagnostics, and medicine.

### BACKGROUND OF THE INVENTION

Real-time polymerase chain reaction (PCR) is the gold standard for the detection of nucleic acids, especially in a diagnostic context (Syvanen, 2001; Fan, et al., 2006). An important problem for both research applications and molecular diagnostics is discrimination between closely related alleles of genes (Williams, 2001; Lyon, et al., 2012; Flegal, 2000). Unfortunately, most real-time assays rely heavily on extensive sample preparation and detailed analysis in machines that detect the number of cycles required for amplification (McGuigan & Ralston, 2002). The presence of impurities or contaminants in samples can lead to non-specific amplification and increasing difficulties in discriminating between alleles (Opel, et al., 2010). In order to better adapt PCR methods, including real-time PCR, for point-of-care applications, it would be desirable to be able to robustly discriminate between alleles, irrespective of sample provenance, condition, preparation, or purity. Allele discrimination via PCR commonly relies upon the use of allele-specific specific primers (Kwok, 2001). General primers can also be used for amplification, and amplicons then probed by single base extension with unlabeled or fluorescently-tagged dideoxynucleotides, ultimately leading to products that are distinguished based on mass (in matrix-assisted laser desorption/ionization time-of-flight) (Jurinke, et al., 2004) or fluorescence (Kim & Misra, 2007).

Allele-specific primers typically contain mismatches at their 3′ ends (so-called ARMS, amplification refractory mutation SNP primers) (Newton, et al., 1989). In real time PCR with allele specific primers there is a delay in amplification of the mismatched target, typically of 5 to 10 PCR cycles, often detected via a so-called TaqMan probe in which a fluor:quencher pair is separated by the exonuclease activity of the polymerase (Livak, et al., 1999). However, certain mismatches are efficiently extended, leading to inaccurate genotyping (Ayyadevara, et al., 2000; Huang, et al., 1992). Improved discrimination against mismatches (a delay in amplification of 5 or 6 cycles) has been reported using locked nucleic acid nucleotides (LNAs) at the 3′ end of a primer, overlapping the mismatch. It has been postulated that the increased melting temperature of LNAs correctly paired with a DNA target resulted in a greater differential in melting temperatures (Latorra, et al., 2003). SNP-specific hairpin primers have also been designed (Hazbon & Alland, 2004). In many cases the hairpin is also a molecular beacon that is triggered when the single-stranded loop sequence hybridizes to the primer-binding site. Scorpion SNP primers are specialized hairpin primers engineered with a full-length linear ARMS primer appended to the 3′ end of a hairpin probe. These primers can be used for the real-time amplification and detection of specific targets via end-point fluorescence (EPF) rather than Cq (quantification cycle). Five nanograms of human genomic DNA amplified with Scorpion primers through forty cycles was sufficient for detection and genotyping of a BRCA2 SNP (Whitcombe, et al., 1999). Non-fluorescent hairpin primers with a single-stranded targeting loop sequence and a SNP-specific nucleotide at the 3′ position in the stem also improved the mean cycle difference between matched and unmatched templates in SybrGreen qPCR assays from 7.6 for linear primers to 11.2 for hairpin primers (Kostrikis, et al., 1998), presumably because of the competition between correct inter- and intramolecular pairing.

The field of nucleic acid computation frequently relies on programming DNA molecules as kinetic traps, which undergo conformational rearrangements upon interactions with input molecules, leading to the execution of algorithms (Benenson, 2012; Chen & Ellington, 2010). One of the chief features of the kinetically trapped nucleic acid substrates is the presence of a short so-called toehold sequence that can initiate strand displacement reactions (Yin, et al., 2008; Srinivas, et al., 2013). In the present disclosure, these principles were applied to the design of hairpin primers that have an initiating toehold sequence that is exquisitely sensitive to mismatches. In the presence of the correct toehold, both strand displacement and elongation can lead to productive amplification of particular SNPs, and discriminate with high fidelity against single mismatches.

### BRIEF SUMMARY OF THE INVENTION

Methods and compositions of the disclosure concern discrimination of alleles in nucleic acid samples using particular strand exchange hairpin primers. The design of the primers allow discrimination between alleles in highly related sequences using a small complementarity sequence harboring a single nucleotide mismatch.

Embodiments of the disclosure include methods and compositions for analysis of nucleic acid(s) by a particular primer. In specific embodiments, there are methods and compositions for analysis of one or more sequences in a nucleic acid by a particular primer. Particular disclosure is provided for methods and compositions for assaying for the presence or absence of a specific sequence or nucleotide in a nucleic acid using a particular primer. Although any specific sequence or nucleotide may be assayed for with methods and compositions of the disclosure, in particular embodiments the specific sequence is a single nucleotide polymorphism (SNP). In specific embodiments, a particular hairpin primer is utilized for allelic discrimination.

Embodiments of the disclosure include methods for primer design and the resultant primers that yield large discrimination between otherwise highly related sequences. Such primers are useful for molecular diagnostics of any kind, such as between a wild-type and drug-resistant allele of an organism or as a marker for a medical condition or risk thereof.

Compositions and methods of the disclosure relate to nucleic acids that target other nucleic acids over a short region, such as a primer that targets a template and initially binds over a short region (such as from 3-15 nucleotides, in at least some cases). In particular aspects, the short region of the hybridization between primer and template is conducive for disruption of the binding if a single mismatch is present in the region. After binding over the short region, two events occur: strand displacement that leads to additional primer-binding, and polymerase extension from the 3′ end of the primer. Binding of the short template-binding region of the primer to the template (or lack thereof) provides huge discriminatory factors that would not be evident if a larger binding region were employed.

Particular primers of the disclosure include those with a hairpin configuration and comprise a toehold (single stranded 3′ end) that allows them to begin to initiate the process of polymerization along with unfolding of the hairpin in the primer.

Particular aspects of the disclosure encompass allelic discrimination using mechanism(s) that go beyond purely thermodynamic discrimination between perfectly paired and mismatched sequences.

The methods and compositions of the disclosure provide high discrimination between closely related genes for qPCR and other types of amplification reactions, including for use in molecular diagnostics. Methods and compositions concern hairpin strand exchange primers in an amplification reaction, including at least qPCR, isothermal amplification reaction, ICAN, NASBA, RPA, RCA, HAD, SDA, LAMP, CPA, EXPAR, and SMAP2.

The methods and compositions of the disclosure provide a degree of allelic discrimination in orders of magnitude greater than any known primers, such as up to 100,000-fold, compared to 10- to 30-fold with hairpin or energy-balanced primers, for example.

Embodiments of the disclosure allow a yes/no evaluation of the presence or absence of a given gene sequence. In some embodiments, the toehold hairpin primers of the disclosure are paired with another type of primer. In particular aspects, the toehold hairpin primers are utilized with normal, nested primers. In particular embodiments, methods and compositions utilize a toehold region on a primer that could at once allow both extension and strand exchange, including in a way that is competitive with respect to single mismatches (i.e., in the presence of mismatches the primer is more likely both to not strand exchange and to not be extended.)

In some embodiments, methods of the disclosure are utilized to assay the presence or absence of an unknown mutation (including an unknown SNP). In several instances, such as cancer-related genes or pathogen drug resistance genes, for example, mutation hotspots are known to exist on these genes. For example, the rpoB gene of Mycobacterium tuberculosis has an 81 bp region called the rifampin resistance determining region that usually contains SNPs in rifampin-resistant bugs. The actual identity or exact location of the SNP within this region can be varied. Primers directed at invariant regions such as 16S rDNA would allow bacterial identification. However, failure/alteration of toehold primer amplification efficiency directed at regions within the mutation hotspot would allow one to detect mutant bacteria even prior to knowing the exact mutation.

In embodiments of the disclosure, there is a composition comprising a single stranded primer, said primer comprising a 5′ end, a region of intramolecular complementarity, and a single stranded 3′ end, wherein the single stranded 3′ end comprises at least one designed mismatched nucleotide in relation to a corresponding region of a nucleic acid to which it is complementary. In certain cases, the single stranded 3′ end is between 3 and 15 nucleotides in length. In specific cases, the primer is at least 18 nucleotides in length, although in some cases the primer is between 18 and 60 nucleotides in length. In a particular embodiment, the primer has a G/C percentage of 40% to 70%. In particular instances, the region of intramolecular complementarity is at least 5 nucleotides in length, although in some cases the region of intramolecular complementarity is between 5 and 50 nucleotides in length.

Primer compositions of the disclosure may further comprise a single stranded loop sequence, such as one that is at least 4 nucleotides in length, although it may be between 4 and 40 nucleotides in length. In certain aspects, the loop sequence comprises homopolymeric sequence, such as all thymidines. Certain primers will have loop sequence that comprises random sequence. In particular embodiments, the loop sequence is specific for a target sequence. Loop sequences may comprise one or more modifications.

In some embodiments, one or more modifications comprise a polymerase-extension blocking moiety, a probe, or a reporter.

In particular aspects of the primer, the designed mismatched nucleotide is present in the primer at the 3′-most nucleotide of the 3′ single stranded end, although the designed mismatched nucleotide may be present in the primer other than at the 3′-most nucleotide of the 3′ single stranded end.

Primers of the disclosure may comprise a label, such as one that is fluorescent, radioactive, or colored. The label is biotin, a protein, a peptide, a nanoparticle, or a crystal, in some cases.

Embodiment of mismatched nucleotides include those that correspond to a known single nucleotide polymorphism in the nucleic acid or those that correspond to a known wild-type nucleotide in the nucleic acid.

In certain embodiments of the disclosure, there is a nucleic acid complex, comprising a primer, said primer comprising a 5′ end, a region of intramolecular complementarity, and a single stranded 3′ end; and a double stranded nucleic acid having a template strand and a complementarity strand, wherein said single stranded 3′ end of the primer is complementary to and bound to a region of a corresponding template strand of the double stranded nucleic acid except for one mismatched nucleotide, and wherein the region of complementarity between the primer and template strand is sufficiently short such that upon binding of the primer to the template strand, there is strand displacement of the complementarity strand from the double stranded nucleic acid and there is polymerization from the 3′ end of the primer when in the presence of a polymerase. The region of complementarity between the primer and template strand may be between 3 and 15 nucleotides in length.

In certain cases, nucleic acid complexes of the disclosure are comprised in a vessel (such as a tube or syringe) or on a substrate (such as a microtitre plate, bead, paper, or slide).

Double stranded nucleic acids of the complex may be from a sample from an individual, such as a mammal, bird, plant, microbe, or virus. The sample may be blood, urine, saliva, biopsy, cheek scrapings, nipple aspirate, cerebrospinal fluid, plasma, fecal matter, sputum, or hair.

In the complex, the primer may comprise a label, including one that is fluorescent, radioactive, or colored. The label may be biotin, a protein, a peptide, a nanoparticle, or a crystal. In the complex, the mismatched nucleotide between the primer and the template strand may be at the site of a single nucleotide polymorphism. In certain aspects, the mismatched nucleotide between the primer and the template strand is at a site suspected of having a single nucleotide polymorphism. In particular embodiments, the single nucleotide mismatch is present in the complex based on design of the primer.

In some embodiments, there is a method of determining the presence or absence of a known nucleotide or known nucleic acid sequence in a sample from an individual, comprising the steps of exposing a primer to nucleic acid from the sample, wherein said primer comprises a 5′ end, a region of intramolecular complementarity, and a single stranded 3′ end, wherein the primer binds to nucleic acid from the sample at a region of complementarity between the single stranded 3′ end and the nucleic acid, wherein when there is a single nucleotide mismatch in the region of complementarity between the single stranded 3′ end of the primer and the nucleic acid, the primer is not able to be polymerized from its 3′ end and no detectable polymerization product is produced, and wherein when there is not a single nucleotide mismatch in the region of complementarity between the single stranded 3′ end of the primer and the nucleic acid, the primer is able to initiate strand displacement and initiate polymerization from its 3′ end and a detectable polymerization product is produced.

In some embodiments, the primer is designed to include the single nucleotide mismatch in the region of complementarity between the single stranded 3′ end of the primer and the nucleic acid. The presence of the known nucleotide or nucleic acid sequence in the sample may be reflected in there being no detectable polymerization product. In some cases, the absence of the known nucleotide or nucleic acid sequence in the sample is reflected in there being no detectable polymerization product, whereas in some cases, the presence of the known nucleotide or nucleic acid sequence in the sample is reflected in there being a detectable polymerization product. In particular embodiments, the absence of the known nucleotide or nucleic acid sequence in the sample is reflected in there being a detectable polymerization product.

In certain aspects of the method, the known nucleic acid sequence comprises a mutation. The known nucleic acid sequence may comprise a single nucleotide polymorphism (SNP).

In specific embodiments of the method, the individual is in need of diagnosis of a medical condition and the presence or absence of the known nucleic acid sequence is indicative thereof. In some cases, when the individual is diagnosed as having the medical condition, the individual is given an effective amount of an appropriate therapy for the medical condition. In certain instances, when the individual is diagnosed as not having the medical condition, the individual is not given a therapy therefor.

In particular aspects of the method, the individual is in need of determination of efficacy of a therapy for the individual and the presence or absence of the known nucleic acid sequence is indicative thereof. In some cases, when the individual is determined to be suitable for the efficacy of the therapy, the individual is provided an effective amount of the therapy. In other cases, when the individual is determined not to be suitable for the efficacy of the therapy, the individual is provided an effective amount of an alternative therapy.

Methods of the disclosure may further comprise the step of obtaining sample from the individual.

In certain embodiments, there is a method of assaying for the presence or absence of a known nucleotide or known nucleic acid sequence in a sample from an individual, comprising the steps of assaying for the presence of a polymerization product from a primer bound to a nucleic acid template at a region of complementarity in the template, wherein the region of complementarity comprises the known nucleotide or known nucleic acid sequence in the template and wherein the primer is bound thereto at its single stranded 3′ end, wherein the region of complementarity is no more than 15, 14, 13, 12, 11, 10, 9, 8, 7, 6, 5, or 4 nucleotides in length, wherein when there is a mismatch in the region of complementarity between the primer and the nucleic acid template, no polymerization product is produced and the presence or absence of the known is determined, or wherein when there is no mismatch in the region of complementarity between the primer and the nucleic acid template, a polymerization product is produced and the presence or absence of the known is determined. In specific aspects, the primer is designed to have a single nucleotide mismatch in the region of complementarity. In certain embodiments, the primer is designed to have no mismatches in the region of complementarity. In particular aspects, the primer is further defined as having a region of intramolecular complementarity and a single stranded loop.

In particular embodiments, nucleic acid capture is achieved by exposing a plurality of nucleic acids to a toehold hairpin primer affixed to a substrate, such as a bead, wherein binding of the primer to nucleic acids to which it is complementary allows capture of such nucleic acids. Following this, the captured nucleic acids may be further processed, such as amplified, including with or without the toehold hairpin primer.

In one embodiment, there is a method of capturing one or more desired nucleic acids from a plurality of nucleic acids, comprising the steps of: exposing a primer-bound substrate to a plurality of nucleic acids, wherein said primer comprises a 5′ end, a region of intramolecular complementarity, and a single stranded 3′ end, wherein the primer binds to nucleic acid from the sample at a region of complementarity between the single stranded 3′ end and the nucleic acid, wherein when there is a single nucleotide mismatch in the region of complementarity between the single stranded 3′ end of the primer and the nucleic acid, the primer is not able to be polymerized from its 3′ end and no polymerization product is produced, and wherein when there is not a single nucleotide mismatch in the region of complementarity between the single stranded 3′ end of the primer and the nucleic acid, the primer is able to initiate strand displacement and initiate polymerization from its 3′ end and a polymerization product is produced; and subjecting said polymerization product to processing, such as amplification, including polymerase chain reaction. In specific embodiments, the amplification utilizes the primer. In certain embodiments, the plurality of nucleic acids comprises nucleic acid from one or more cells, such as from an individual, and the individual may be suspected of having or being at risk or susceptible to a particular medical condition. In specific embodiments, the substrate is a microtitre plate, bead, paper, or slide. In certain embodiments, the region of complementarity between the single stranded 3′ end of primer and the nucleic acid is no more than 15, 14, 13, 12, 11, 10, 9, 8, 7, 6, 5, or 4 nucleotides in length.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is a schematic of toehold-dependent strand displacement primers for enhanced SNP distinction. The objective is to find the minimum toehold that is stable enough to bind the target and initiate strand displacement. Any mismatch in the toehold will disrupt priming and amplification.

FIG. 2A shows real-time assays using KatG WT THPs reveal greatly reduced (i.e. T5 or T6 primers) or no amplification (i.e. T4 primers) of unmatched templates when compared with analogous linear primers. FIG. 2B shows that the KatG WT T4 primer does not amplify the mismatched SNP template. The linear primer (i.e. lin) discriminates poorly in comparison with a ΔCq of 6. FIG. 2C shows that THPs targeting drug resistance SNPs in both of the M. tuberculosis genes tested (KatG and RpoB) demonstrate superior allele specificity and SNP discrimination.

FIG. 3 demonstrates efficiencies and limit of detection of KatG and RpoB THPs were tested with concentrations between 1 ng and 1 pg of plasmid template.

FIG. 4 provides a simple example of a protocol that produced visible bands for the T6 primer at 20 cycles using 1 ng of template: two-step PCR with a 2 min denaturing step at 95° C., and 20 cycles with a 30 s 95° C. denaturing step followed by a 30 s annealing/extension incubation at 68° C.

FIG. 5 shows performance of linear, T4, and T0 (filled toehold) primers with 10 ng of matched template DNA over an annealing gradient between 60° C. and 72° C.

FIG. 6 shows detection of E6 HPV protein in Purified RNA from Hela Cervical Carcinoma Cells. Toehold hairpin and linear primers were conjugated to 1 micron beads for capture.

FIG. 7 shows toehold hairpin primers for mRNA capture on bead. Linear primers were utilized for reverse transcription and the toehold hairpin primers were used for qPCR. Detection of a 1 bp Notch1 SNP was shown in Hela cells vs. WT Notch1 in A431 cells.

FIG. 8 demonstrates human 18S (positive control) bead capture with 300 cells or 9 ng of whole RNA. Linear reverse transcription was followed by toehold hairpin primer qPCR.

FIG. 9 demonstrates Notch1 Hela PPV SNP detection. Bead capture was employed with 300 cells or 9 ng of whole RNA. Linear reverse transcription was followed by toehold hairpin primer qPCR.

### DESCRIPTION OF THE TABLES

Table 1. Multiple drug resistance alleles in M. tuberculosis. Alleles targeted in the THP SNP assays arise as genetic mutations conferring resistance to isoniazid and rifampin in treated human populations.

Table 2. Sequences are provided for the primers detailed in the studies, including common reverse primers, linear control primers, and filled toehold and scrambled stem negative control primers. Fluorescent hydrolysis probes used to detect template-specific amplification products in real-time assays are also shown.

### DETAILED DESCRIPTION OF THE INVENTION

As used herein, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

The term “primer,” as used herein, is meant to encompass any nucleic acid that under appropriate conditions is capable of priming the synthesis of a nascent nucleic acid in a template-dependent process.

The term “toehold” as used herein refers to a single stranded section at the 3′ end of a hairpin primer. In particular aspects, the toehold comprises one or more nucleotides that are mismatched compared to a reference sequence.

### I. General Embodiments

Methods and compositions of the disclosure concern the allelic discrimination of a particular known nucleotide or nucleic acid sequence. The identification of the particular known nucleotide or nucleic acid sequence occurs upon the use of a primer that binds to a corresponding template at a region that includes the known nucleotide or nucleic acid sequence and upon the nature of the efficiency of the primer binding and its ability to be extended by a suitable polymerase. In particular embodiments, the known nucleotide whose identify is in question is a single nucleotide polymorphism (SNP). The identity of the SNP is desired for research or medical purposes.

The ability to detect and monitor SNPs in biological samples is an enabling research and clinical tool. The disclosure encompasses a surprising, inexpensive primer design method that provides exquisite discrimination between single nucleotide polymorphisms, for example. The field of DNA computation is largely reliant on using so-called toeholds to initiate strand displacement reactions, leading to the execution of kinetically trapped circuits. The present disclosure demonstrates that the short toehold sequence to a target of interest can initiate both strand displacement of the hairpin and extension of the primer by a polymerase, both of which will further stabilize the primer:template complex. However, if the short toehold does not bind, neither of these events can readily occur. As described below, toehold hairpin primers were used to detect drug resistance alleles in two exemplary genes, rpoB and KatG, in the Mycobacterium tuberculosis genome. During real-time PCR, the primers discriminate between mismatched templates with delta Cq values that are frequently so large that the presence or absence of mismatches is essentially a qualitative answer, such as a ‘yes/no’ answer. Methods and compositions of the disclosure provide broad use for allele detection, especially in point-of-care settings where yes/no answers are most valued.

The disclosure provides a set of primer design principles and a toolkit of primers that distinguish SNPs with a very high degree of discrimination. Such primers find application in diagnosis of metabolic and infectious diseases where SNPs serve as biomarkers of the disease or the pathogen.

### II. Primers and Primer/Template Complexes

A. Primers

The disclosure concerns primers that are utilized to determine the presence or absence of a known nucleotide or nucleic acid sequence using high allelic determination. The design of the primers are such that their ability to be polymerized from the 3′ end is indicative of whether or not a particular nucleotide or nucleic acid sequence is present in a template to which it binds. In some cases, the ability to by extended at its 3′ end indicates whether there is a certain nucleotide or nucleic acid sequence in a template to which it binds, whereas in other cases the absence of the ability to be extended at its 3′ end indicates whether there is a certain nucleotide or nucleic acid sequence in a template to which it binds.

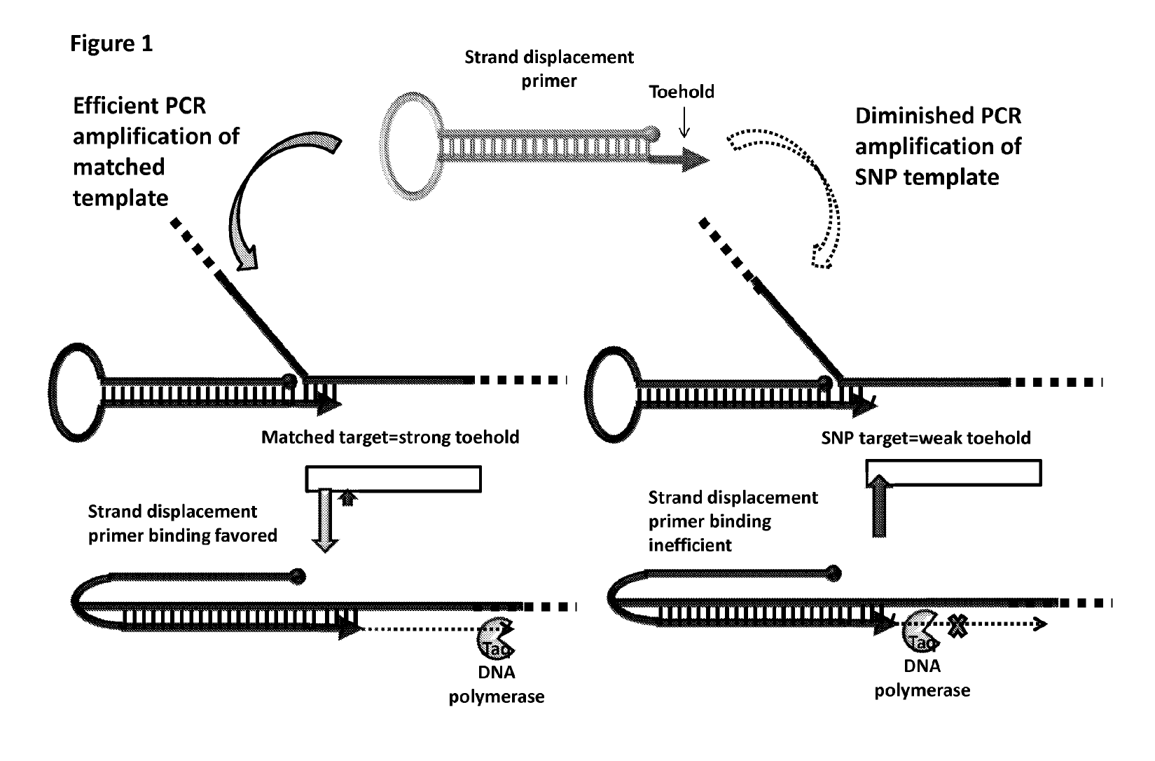

In particular aspects, the primer comprises particular characteristics. In certain embodiments, the primer comprises a hairpin (a region of intramolecular complementarity). The primer may have one or more regions that are single stranded. In some cases, a single stranded loop is present, for example in a configuration that interrupts the strand at the region of the intramolecular complementarity of the hairpin (see FIG. 1). The primers comprise a 3′ end that is single stranded in nature, and the relative shortness of the single stranded end allows such primers to be referred to as toehold primers. The single stranded 3′ end of the primers comprises a nucleotide that is intentionally designed based on an expected nucleotide or sequence of nucleotides in a corresponding template to which the 3′ end binds. The design may be such that it is intended to be mismatched to the particular nucleotide or sequence of nucleotides in the corresponding template. In some cases, the design may be such that it is intended not to be mismatched to the particular nucleotide or sequence of nucleotides in the corresponding template.

The primers, in specific aspects, may comprise in a 5′ to 3′ direction: a 5′ end, a first strand of intramolecular complementarity, a single stranded loop, a second strand of intramolecular complementarity that is complementary to the first strand of intramolecular complementarity and bound thereto, and a single stranded 3′ end. The lengths and/or content of each region of the primer may be of any suitable kind, although in some cases the lengths and/or content of each region is of a particular nature.

For example, in some cases, the region of intramolecular complementarity may be of any suitable kind but may comprise at least 5 paired nucleotides, or it may be in a range of length of nucleotides, such as between 5 and 50 nucleotides. The region of intramolecular complementarity is useful to prevent premature binding of those regions to a template, in at least some cases. In particular embodiments, the 5′-most end of the primer is part of the region of intramolecular complementarity. The single stranded loop may be of any length and nucleotide sequence, but in particular cases it is sufficiently long so that the second strand of intramolecular complementarity may be able to bind the first strand of intramolecular complementarity at the appropriate sequence. In specific embodiments, the single stranded loop is at least 4 nucleotides in length, although in some cases it is 4-8 nucleotides, but in certain embodiments it is up to and including 40 nucleotides in length. The nature of the sequence of the loop may be of any kind. In specific embodiments, the loop sequence employs target-non specific loop sequences. In certain cases, the loop sequence is tailored with a variety of sequences, such as homopolymeric loops or random sequences to achieve desired energetic and structural properties of the primers. In some cases, the loop is designed to be specific to a target sequence. The loop might also be designed to contain one or more modifications, including polymerase-extension blocking moieties, such as ethylene glycol spacers, probes, or reporters. In some cases, the loop is comprised of all thymidines or the majority are thymidines.

The single stranded 3′ end of the primer, which may be referred to as the toehold, is a region of particular length so that it is short enough such that any equilibration that occurs between the single stranded 3′ end and the target sequence in the template would be greatly affected by any mismatch between the single stranded 3′ end and the corresponding target sequence in the template. In specific embodiments, the single stranded 3′ end is wholly complementary to the corresponding target sequence except for one nucleotide, although in certain cases the single stranded 3′ end is wholly complementary to the corresponding target sequence in the template. In specific embodiments, the 3′ end is between 3 and 9 nucleotides or between 3 and 15 nucleotides in length. In some embodiments, the single stranded 3′ end can be longer than 15 nucleotides (such as 16, 17, 18, 19, 20, 21, 22, 23, 24, 25, 26, 27, 28, 29, 30, 35, 40, 45, 50 or more nt in length) and contain 1, 2, 3, 4, 5, 6, or more additional destabilizing mismatches in addition to the SNP specific mismatch.

The primer may be of a particular G/C percentage (such as between 40% and 70%), although in at least some cases the nature of the sequence in the template surrounding the particular nucleotide or nucleic acid sequence will dictate the percentage of G/C in the corresponding primer.

In some cases, the primer may be labeled, and such a label may be of any suitable type in the art so long as it allows the primer or an extension product therefrom to be detectable, such as by the naked eye or by machine. In specific embodiments, the label is fluorescent, colorimetric, or radioactive.

In embodiments, the primers are intentionally designed by the hand of man to include a mismatch with a template sequence or are intentionally designed not to include a mismatch with a template sequence, rather than having or not having the mismatch based on chance.

B. Primer/Template Complexes

Embodiments of the disclosure include a complex between a primer as described herein and a nucleic acid template to which it is complementary and able to bind at least in part. The nucleic acid may be obtained from a plurality of other nucleic acids and therefore substantially isolated, although in some cases the primer is able to recognize the nucleic acid template among a plurality of other nucleic acids. In its natural state, the nucleic acid template is configured in a double stranded manner, with the nucleic acid template bound to its complementary strand.

In embodiments of the disclosure, there is a nucleic acid complex, comprising a toehold hairpin primer and a target double stranded nucleic acid having a template strand and a complementarity strand. A single stranded 3′ end of the primer is complementary to and bound to a region of the corresponding template strand of the double stranded nucleic acid with the exception of one mismatched nucleotide. The region of complementarity between the primer and template strand may be sufficiently short such that upon binding of the primer to the template strand, there is strand displacement of the complementarity strand from the double stranded nucleic acid and the 3′ end of the primer is extendable.

The primer/template complex may be among a plurality of primer/template complexes in situations where there is no mismatch between the primer and the template strand and the 3′ end is extendable.

### III. Exemplary Applications for the Methods

Methods of the invention allow allelic discrimination based on mismatch discrimination that relies on equilibration of a very small sequence, leading to strand displacement that allows further primer binding and strand extension from the primer. The methods allow discrimination at a particular nucleotide or nucleic acid sequence, although in particular cases the methods are employed to allow identification whether or not a particular SNP is present.

In embodiments, a strand displacement primer comprising a toehold is provided to a nucleic acid template, wherein there is perfect complementation between the primer and the template at the entire sequence of the toehold. The perfectly matched target provides a strong toehold that allows primer binding, resulting in efficient polymerization (such as with PCR amplification, for example). However, in cases wherein there is a mismatch in the toehold region of the primer, there is a weak toehold leading to inefficient primer binding, resulting in a diminished polymerization (such as with PCR amplification, for example). In specific embodiments when there is inefficient binding between the toehold hairpin primer and the template, it is because the template comprises the SNP (in the region of the template that is complementary to the toehold region of the primer) and therefore there is mismatching between the toehold region of the primer and the template. Such a mismatch between the primer and the template leads to inefficient amplification, and in this particular case the absence of a PCR product is indicative of presence of the SNP in the template. Thus, the rationally designed SNP-distinguishing primers hybridize to the correct (complementary) templates with a much greater efficiency, while binding to templates comprising a single nucleotide change is greatly diminished. This establishes a large amplification bias in favor of the correct template versus the SNP-containing template, allowing accurate alleleic distinction in real time.

The presence or absence of a particular SNP or nucleic acid sequence may be determined based on a number of designs of the methods. That is, the presence or absence of a SNP may be determined upon identification of efficient amplification in the method, or the presence of absence of a SNP may be determined upon identification of inefficient amplification in the method. For example, in some cases, a primer is designed such that it will have a mismatch compared to a known nucleotide in the template, and the absence of polymerization from the primer 3′ end in this case (which may be visualized based on absence of amplification by PCR) confirms the identity of the known nucleotide. In some cases, a primer is designed such that it will not have a mismatch compared to a known nucleotide in the template, and the presence of polymerization from the primer 3′ end in this case (which may be visualized based on presence of amplification by PCR) confirms the identity of the known nucleotide.

In certain aspects, the identity of a particular nucleotide is suspected, and the identity of the nucleotide is confirmed or refuted based on the ability of a particular primer to be polymerized from its 3′ end. For example, an individual may be suspected of having a particular SNP. A primer is designed that either is or is not mismatched compared to the identity of the suspected SNP nucleotide. Upon performing the method, in cases when the primer is designed to be mismatched with the suspected SNP nucleotide, no polymerization product is produced, and the absence of polymerization product informs one that the individual has the corresponding suspected SNP nucleotide. In this same example, in cases when the primer is designed to be mismatched with the suspected SNP nucleotide, and when a polymerization product is produced, this informs one that the corresponding suspected SNP nucleotide is not present in the individual. In cases when the primer is designed not to be mismatched with the suspected SNP nucleotide, a polymerization product is produced, and the presence of the polymerization product informs one that the individual has the corresponding suspected SNP nucleotide. However, in this example, in cases when the primer is designed not to be mismatched with the suspected SNP nucleotide and a polymerization product is not produced, the absence of the polymerization product informs one that the individual does not have the corresponding SNP nucleotide.

Thus, in specific embodiments there is a method of determining the presence or absence of a known nucleotide or known nucleic acid sequence in a sample from an individual. A primer is exposed to nucleic acid from the sample and when there is a single nucleotide mismatch in the region of complementarity between the single stranded 3′ end of the primer and the nucleic acid, the primer is not able to be polymerized from its 3′ end and no detectable polymerization product is produced, yet when there is not a single nucleotide mismatch in the region of complementarity between the single stranded 3′ end of the primer and the nucleic acid, the primer is able to be polymerized from its 3′ end and a detectable polymerization product is produced. In some cases, the presence of the known nucleotide or nucleic acid sequence in the sample is reflected in there being no detectable polymerization product. In other cases, the absence of the known nucleotide or nucleic acid sequence in the sample is reflected in there being no detectable polymerization product. In specific cases, the presence of the known nucleotide or nucleic acid sequence in the sample is reflected in there being a detectable polymerization product, although in certain aspects the absence of the known nucleotide or nucleic acid sequence in the sample is reflected in there being a detectable polymerization product.

In particular embodiments, an individual is in need of determination whether or not a nucleic acid in their cells comprises a particular nucleotide or nucleic acid sequence. In some cases, the presence or absence of the particular nucleotide or nucleic acid sequence in nucleic acid in a sample from the individual is indicative of the presence of a particular medical condition, indicative of the effectiveness of a particular therapy for a medical condition that the individual is known to have, is predictive whether or not an individual is at risk for having a particular medical condition, and so forth. In some cases, the method is employed for paternity testing. The methods of the invention provide utility whether or not the individual is determined to have or at risk of having a medical condition or whether or not a therapy will be effective for the individual. The individual in need provides a sample that comprises nucleic acid to be analyzed, and the medical condition in question will determine what sample is suitable. In some cases, the sample comprises blood, plasma, serum, biopsy, saliva, urine, cheek scrapings, nipple aspirate, cerebrospinal fluid, fecal matter, hair, and so forth. The nucleic acid may be isolated from cells in the sample. The nucleic acid may be further manipulated prior to analysis, such as to remove associated proteins, to remove RNA, and so forth. In some cases, the individual performing the method(s) of the disclosure also is the individual that obtains and/or processes the sample, although in other cases a third or more party obtains the sample from the individual and/or processes it.

In particular cases, the methods are employed in a point-of-care situation, where a sample from an individual is in need of being assayed when the individual is present and, in some cases, has freshly provided a sample for analysis. In particular embodiments, the point-of-care situation is in a doctor's office, hospital, combat zone, school, cruise ship, hotel, sports facility or clubhouse, managed care facility, old age homes, nurseries, camps, and so forth.

Embodiments of the disclosure include methods of treatment for the individual. For example, in some cases, an individual is provided an effective amount of a suitable treatment when the individual is determined to have a medical condition based on the results of methods of the invention, an individual is provided an effective amount of a suitable treatment when the individual is determined to be susceptible to a medical condition (or preventative action therefor), and an individual is provided an alternative therapy when the methods of the disclosure identify the individual as being unsatisfactory to receive a particular therapy (or is provided the therapy when it is determined that it can be effective).

The primer design principles and primer sets provided herein can distinguish SNPs with up to 100,000-fold degree of discrimination. This makes alleleic discrimination more reliable with a yes/no level of accuracy.

### IV. Nucleic Acid Capture

In particular embodiments, one or more desired nucleic acids are captured from a plurality of nucleic acids. The desired nucleic acids may be obtained from among the plurality of nucleic acids that includes them. In particular embodiments, the desired nucleic acids are captured upon binding to complementary toehold hairpin primers as contemplated herein.

In certain embodiments, the toehold hairpin primers are affixed to a substrate to form a primer-substrate entity and the primer-substrate entity is subjected to a plurality of nucleic acids that is known to comprise or suspected of comprising particular nucleic acids of interest that are complementary to at least part of the primers. In specific embodiments, the region of complementarity is no longer than a particular size, such as no longer than 15, 14, 13, 12, 11, 10, 9, 8, 7, 6, 5, or 4 nucleotides in length. In certain embodiments, the region of complementarity between the toehold hairpin primer and the desired nucleic acids comprises a mismatch. The mismatch may be designed in the primer. The primers may be affixed to a substrate, such as a solid surface. In specific embodiments, the substrate comprises a slide, bead, tube, column, cylinder, or plate.

In particular aspects of the disclosure, certain nucleic acid molecules are targeted by toehold hairpin primers that are conjugated to substrates such as beads. The nucleic acids may include all nucleic acids present in an organism, including cell free fetal DNA in pregnancy, DNA fragments in the blood of tumor patients, mRNA and microRNA, long noncoding RNA, and snoRNA, in cells and body fluids, or RNA or DNA fragments from viral or bacterial pathogens that are present in the organism. A plurality of nucleic acid molecules, such as from one or more cells, one or more samples, or one or more cells from one or more samples, are exposed to beads having the designed toehold hairpin primer of interest conjugated thereto. In cases wherein the plurality of nucleic acids to be assayed comes from cells, the cells may be lysed and the nucleic acids may be extracted therefrom. In some embodiments, the desired nucleic acids are particular mRNAs. The desired nucleic acids may be suspected of having one or more particular SNPs. The plurality of nucleic acids may be from an individual suspected of having, being at risk for, or being susceptible to a particular medical condition, and the medical condition may or may not be related to the presence of one or more SNPs.

In specific cases a single substrate, such as a bead, comprises multiple primers conjugated thereto. In certain embodiments the primer is conjugated to the substrate via the 5′ end of the primer so that the 3′ end is available for complementation to an appropriate and desired nucleic acid.

Upon exposure of the primer-substrate entity to the plurality of nucleic acids to be assayed for the desired nucleic acids therein, the desired nucleic acids bind the primer at the region of complementarity. In some cases, there is a mismatch in the region of complementarity and strand displacement cannot be initiated and polymerization from the 3′ end of the primer cannot occur to any appreciable extent. In other cases there is not a mismatch in the region of complementarity between the single stranded 3′ end of the primer and the desired nucleic acid, the primer is able to initiate strand displacement and initiate polymerization from its 3′ end and a polymerization product can be produced.

Upon capture of the desired nucleic acids from the plurality, those nucleic acids that did not hybridize to the primer may be washed away from the primer-substrate entities by standard means in the art.

Upon capture of the desired nucleic acids, the nucleic acids may be further processed. Such applications may include reverse transcription, amplification, visualization, enzyme digestion, cloning, sequencing or combinations thereof. Particular embodiments include mRNA and miRNA and the captured desired mRNA or miRNA is reverse transcribed and amplified by quantitative real time polymerase chain reaction, for example.

### V. Single Nucleotide Polymorphisms (SNPs)

In some embodiments, compositions and methods of the disclosure concern identification of the presence or absence of a SNP. SNPs, the most common source of genetic variation among individuals, often serve as biomarkers for diseases, such as cancer, as well as for predicting drug responses and risk of developing diseases. Accurate SNP detection is often also critical for diagnosis and management of infectious diseases, such as tuberculosis where pathogen-associated SNPs result in drug resistance. While several methods of allelic discrimination have been described, none of them afford the almost yes/no extent of discrimination that is observed with the present disclosure. The greatly improved ability to distinguish SNPs using compositions and methods of the present disclosure is especially useful, because most biospecimens comprise alleleic mixtures of genetic material.

In some cases, more than one SNP is assayed from the same sample from an individual, such as wherein the presence or absence of multiple SNPs is informative about a particular medical condition, risk thereof, or effectiveness of therapy thereof. In such cases, more than one toehold hairpin primer may be utilized in methods of the disclosure.

In particular aspects, once the presence or absence of a SNP has been identified from methods of the disclosure, the region of the SNP may be further assayed, such as by sequencing, for example.

Although any SNP may be identified with methods and compositions of the disclosure, in some cases the SNP is associated with cancer, tuberculosis, malaria, pathogen typing, including drug resistance, or risk for a medical condition or efficacy of treatment for a medical condition. Examples of SNPs associated with tuberculosis include KatG S315T or RpoB Q513L.

In a particular example, a SNP in the TNFR (tumor necrosis factor receptor) II gene is indicative of rheumatoid arthritis. In another particular example, the TNFR2 polymorphism or other genetic variations in tumor necrosis factor or related genes is indicative of suitable familial rheumatoid arthritis treatment response to TNF inhibitors.

As catalogued in the HapMap project and NCBI's SNP database dbSNP, single nucleotide polymorphisms are one of the most common type of human genetic variation. These variations have been associated with diseases such as thalassemia, cystic fibrosis, sickle-cell anemia and breast cancer; population diversity; susceptibility to infectious agents such as HIV and Mycobacterium tuberculosis; and individual response to medicine. Hence, SNP genotyping has become an important tool for determining disease susceptibility, pharmacokinetics and diagnostics. A very small example set of such SNPs include: Adrenoreceptor β 2 G16R G>A (rs1042713) and Nitric oxide synthase D298E T>G (rs1799983) for arterial hypertension; Hypoxia induced factor 1 alpha P582S C>T (rs11549465) and Apolipoprotein E C112R T>C (rs429358) for ischemic heart disease; ATP-sensitive inward rectifier potassium channel E23K C>T (rs5219) and Transcription factor PPAR gamma P12A C>G (rs1801282) for diabetes mellitus type 2 and; Vascular endothelial growth factor receptor 2 Gln472His T>A (rs1870377) and Vascular endothelial growth factor A 4534C>T (rs833061) for imatinib efficacy.

### VI. Samples

In some aspects for the disclosure, the methods and compositions are utilized for the purpose of analyzing nucleic acid from an individual, such as a mammal (including humans, dogs, cats, horses, etc.) in certain cases, the methods and compositions are employed for plant samples, such as plant identification or crop breeding programs, and for analysis of SNP evolution in microorganisms. Although the nucleic acid may be analyzed for any suitable purpose, in some cases the individual is in need of the analysis for a medical purpose. Any particular medical purpose is applicable for the methods and compositions, but in particular embodiments the individual is in need of diagnostic analysis, prognostic analysis, and/or analysis for the purpose of predicting effectiveness of a therapy. The individual may or may not be known to have a particular medical condition.

In cases wherein methods and compositions are employed for predicting effectiveness of a therapy, the individual may already be receiving the therapy or the individual may not have yet begun receiving the therapy. In some cases, an individual is in need of knowing whether or not they will become resistant to a therapy.

A sample may be obtained from the individual for extraction of nucleic acid, and routine methods are known in the art for nucleic acid extraction from biological samples. The sample may be obtained from the individual by the provider of the method of the invention, or the sample may be obtained from the individual by another party. The sample may or may not be manipulated prior to nucleic acid extraction. The sample may be of any kind so long as nucleic acid is extractable therefrom. In specific aspects, the sample comprises blood, serum, plasma, urine, cerebrospinal fluid, biopsy, nipple aspirate, saliva, sputum, fecal matter, hair, and so forth.

In particular aspects, SNPs are identified as a marker related to disease or normal traits. SNPs may be assayed for to determine whether or not a certain drug will act in an individual, including for whether or not the target for the drug therapy is present or whether or not the drug would be properly metabolized. Certain diseases may be assayed for, including at least sickle-cell anemia, β-Thalassemia, cancer (including breast cancer), phenylketonuria, muscular dystrophy, Crohn's disease, cystic fibrosis, and so forth.

### VII. Amplification Methods

In embodiments of the methods of the invention, the ability of polymerization to occur from the 3′ end of the hairpin primer(s) of the invention (also referred to as “extension”) is determined and is indicative of the identity of a particular nucleotide or nucleic acid sequence in an nucleic acid. The polymerization may occur as part of a polymerase chain reaction (PCR).

The particular polymerization conditions of the method may be of any kind so long as the 3′ end of the primer may be extended if no mismatch is present between the primer and its template and so long as the 3′ end of the primer would not be extended if a mismatch was present between the primer and its template. Particular salt, temperature, dithiothreitol concentrations, formamide concentrations, and so forth conditions may be optimized per routine practices in the art.

The detection of a product, if present to be detected, may occur by any suitable means. The product may be detected as part of real time PCR, for example. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In specific embodiments, the polymerase employed in the methods is a polymerase that has strand displacement activity. Specific examples of polymerases include at least phi29 polymerase; Bst DNA Polymerase, Large Fragment; Deep VentR™ (exo-) DNA Polymerase; Klenow Fragment (3′→5′ exo-); VentR® (exo-) DNA Polymerase; Bsu DNA polymerase large fragment; Deep Vent; DNA polymerase I Klenow large fragment; or M-MuLV reverse transcriptase.

### VIII. Kits of the Invention

Any of the compositions described herein may be comprised in a kit. In a non-limiting example, one or more primers of the disclosure, polymerization reagents, polymerases, nucleic acid extraction reagents, and so forth may be comprised in a kit. The kits will comprise such compositions in suitable container means.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial or tube. The kits of the present invention also will typically include a means for containing the compositions in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention

### Example 1

### Materials and Methods

Oligonucleotides and Plasmid Construction

Oligonucleotides were utilized from Integrated DNA Technologies (IDT, Coralville, Iowa). M. tuberculosis gene segments were PCR amplified using Phusion DNA polymerase (New England Biolabs, NEB; Ipswich, Mass.) from commercially available genomic DNA of the virulent strain H137Rv (ATCC; Manassas, Va.) and gene-specific primers:

(SEQ ID NO: 1)
KatG Forward: TGGGCGGACCTGATTGTTTTCGCCGGC
(SEQ ID NO: 2)
KatG Reverse: GCTCTTAAGGCTGGCAATCTCGGCTTCGCC
(SEQ ID NO: 3)
RpoB Forward: TGCGATCGACGCTGGAGAAGGACAA CACCG
(SEQ ID NO: 4)
RpoB Reverse: TGTAGTCGGCCGA CACCTCCTCGATGACGC

The PCR products were purified from agarose gels using the Wizard SV gel and PCR purification system (Promega; Madison, Wis.). SNP-containing alleles were then built by overlap PCR amplification of the wild-type gene segments using site-specific mutagenic primers. Following A-tailing using Tag DNA polymerase (NEB), the PCR products were TA cloned into a pCR2.1TOPO vector (Life Technologies; Grand Island, N.Y.) and verified by sequencing at the Institute of Cellular and Molecular Biology Core DNA sequencing facility (University of Texas at Austin; Austin, Tex.).

End-Point PCR

End-point PCR assays were performed using 200 μM deoxynucleotides (Thermo Scientific; Pittsburgh, Pa.), 1 ng of cloned plasmid template, and 5 units of Tag polymerase in a 20 μl reaction on an MJ Research PTC-200 Thermal Cycler, 1×PCR buffer consisted of 50 mM KCl, 10 mM Tris-Cl, pH 8.3, and 1.5 mM MgCl₂. Five μl of each PCR reaction were electrophoresed on a 4% SeaKem LE Agarose gel (Lonza; Rockland Me.) with 0.2 μg/ml ethidium bromide and were visualized with a UV lamp. To determine optimal annealing temperatures, gradient temperatures between 55° C. and 68° C. (55° C., 56.1° C., 58.7° C., 62.8° C., 66° C., and 67.8° C.) were tested and analyzed in the following protocol. KatG WT linear primers and THPs with toehold lengths of 0, 3, 4, 5, 6, 7, 8, and 9 nt were used at a concentration of 200 nM in a three-step PCR reaction with the following conditions: 95° C. for 2 min, followed by 30 cycles of 95° C. for 30 s, annealing at the gradient temperatures listed above for 30 s, and extension at 68° C. for 30 s. Preliminary results favored an annealing temperature of 60° C. in a three-step PCR, with no improvement of amplification for toeholds longer than 6 nt (data not shown). Thus, further optimization of T0, T3, and T6 primers was performed with varying annealing times, MgCl₂ concentrations, and steps in the PCR reaction, Reaction conditions were: 95° C. for 2 min, followed by 20 cycles of 95° C. for 30 s, annealing at 60° C. for 30 s or 20 s, and extension at 68° C. for 30 s. Separate reactions with MgCl₂ concentrations of 1.5 mM and 2.5 mM were run. Testing of two-step PCR was initiated with these conditions: 95° C. for 2 min, followed by 20 cycles of 95° C. for 30 s and combined annealing/extension at 68° C. for 30 s or 45 s. Separate reactions with MgCl₂ concentrations of 1.5 mM and 2.5 mM were run.

Real-Time PCR

All real-time PCR assays were performed on the LightCycler 96 System (Roche Diagnostics; Indianapolis, Ind.) in 96-well format with three technical replicates per sample using Fast Universal Probe Master (ROX; Roche) and FAM-labeled hydrolysis probes with an Iowa Black quencher (IDT). THPs with 3, 4, 5, and 6 nt toeholds were tested for each allele. LightCycler 96 software was used to determine the quantification cycle (Cq) and analyze primer efficiencies. For all assays, unless explicitly stated, template concentration was 1 ng of plasmid, primer concentration was 200 nM, and probe concentration was 55 nM. Ramp times were 1.1° C./is for cooling and 2.2° C./s for heating. The default parameters of the LightCycler SW 1.1 software were adopted for all analyses. For KatG THPs, conditions were as follows: 95° C. for 10 min, followed by 45 cycles of 95° C. for 10 s and annealing/extension at 68° C. for 30 s. To initiate the RpoB Q513 SNP assays, we performed a three-step PCR gradient on the LightCycler to determine optimal conditions for amplification with SNP discrimination. This initial amplification reaction was performed with 10 ng of template with reaction conditions of 95° C. for 10 min, followed by 60 cycles of 95° C. for 15 s, annealing between 65° C. and 72° C. for 20 s, and extension at 72° C. for 20 s. A two-step PCR with 95° C. for 10 min followed by at least 45 cycles of 95° C. for 15 s and combined annealing/extension at 72° C. for 30 s was optimal for amplification and discrimination. To quantify primer efficiency and establish a limit of detection, at least three real-time assays each for the KatG WT- and RpoB WT-specific primers were run with template concentrations (in triplicate) of 1 ng, 100 pg, 10 pg, and 1 pg. Efficiencies (E) were calculated as E=10̂(−1/slope of the standard curve).

### Example 2

### Design of Toehold Hairpin Primers

In certain embodiments, one identifies SNPs during real-time PCR amplification such that a SNP-specific primer perfectly binds its matched template and reacts poorly with a mismatched template. In certain cases, an initial discrimination between matched and mismatched primers leads to much more productive amplification of only the matched sets. By manipulating the DNA toehold strand displacement designs originally described in the field of DNA computing, provided herein is a model for mismatch discrimination that relies on equilibration of a very small sequence ‘seed,’ rather than equilibration of a much larger primer. In this model, the initial binding of the seed leads to two processes, which may occur in parallel: first, strand displacement that leads to additional primer-binding and second, strand extension (FIG. 1).

In designing the primers, which may be referred to herein as Toehold Hairpin Primers (THPs), it was clear that there were several variables that would likely impact their performance, including the length and sequence of the toehold, the length of the hairpin, and the placement of mismatches within either the toehold or the hairpin. For example, in a previous study, toehold length was shown to play an important role in toehold-mediated strand-displacement reactions. Changes in the length of the toehold from 5 to 6 nt led to changes in branch migration rates of upwards of 10-fold (Zhang & Winfree, 2009).

Maximum qPCR discrimination was addressed for two common SNPs conferring drug resistance in Mycobacterium tuberculosis: KatG S315T and RpoB Q513L (Table I).

TABLE I
M. Tuberculosis Drug Resistance Alleles

| | | Amino Acid | SNP | Confers |
|---|---|---|---|---|
| Gene | Function | Mutation | Nucleotide | Resistance to: |
| KatG | Catalase | S315T | AGC to ACC | Rifampin |
| | peroxidase | | | |
| RpoB | RNA polymerase | Q513L | CAA to CTA | Isoniazid |
| | B subunit | | | |

Isoniazid susceptibility in M. tuberculosis is mediated by the product of the KatG gene that encodes a heme-containing catalase. A single nucleotide mutation that changes amino acid 315 from serine to threonine is sufficient to confer isoniazid resistance and is a commonly observed mutation in drug resistant tuberculosis infections (Imperiale, et al., 2013; Farooqi, et al., 2012; Heym, et al., 1993; Kiepiela, et al., 2000). The antibiotic rifampin inhibits M. tuberculosis RNA polymerase and resistance frequently arises from mutations in codon 513 of the beta subunit of the polymerase, the RpoB gene (more than 50% of rifampin resistant isolates in some studies [Fan, et al., 2003; Zaczek, et al., 2009]).

In order to promote maximum discrimination between these alleles and their wild-type counterparts, the mismatch was placed within the short toehold region. It was considered that any equilibration that occurred between the short toehold and the target sequence would be greatly affected by the mismatch, preventing either subsequent strand displacement and/or strand elongation by a thermostable polymerase. Further, by using the ARMS strategy of placing the allele-specific nucleotide at the 3′ end of the toehold, one could use the discriminating properties of Tag polymerase, that binds but does not efficiently extend a 3′ mismatched primer (FIG. 1)(Newton, et al., 1989; Huang, et al., 1992). Other polymerase that lack 3′-5′ proofreading ability, such as some of the enzymes referred to above, including Vent(exo-), Deep vent (exo-) and Klenow(Exo-), may be used. Mismatches within but not exactly at the 3′-end of toeholds can also be distinguished.

Two important considerations for determining the stem length were that (i) the sequence of the extended primer was long enough to be specific for the target and that (ii) the hairpin structure remained stable at annealing and elongation temperatures typical of real-time assays (between 60° C. and 72° C.). A stem length of 18 bp was chosen for the KatG target and 19 bp for the RpoB target. The loop sequence for both targets was a stretch of six thymidines. There are no previous studies of the kinetics of toehold-mediated strand-displacement at a high temperature and therefore toehold lengths from 3 to 9 nt (that is, T3 to T9 primers) were initially assessed for single mismatch discrimination. All allele-specific primers shared a common linear reverse primer (Table II).

| | KatG WT Toehold Hairpin | KatG S315T SNP Toehold Hairpin |
|---|---|---|
| | Primers | Primers |
| T0 | CTGGTGATCGCGTCCTTACC | CTGGTGATCGCGTCCTTACCGG |
| | GGTTTTTTCCGGTAAGGACG | TTTTTTCCGGTAAGGACGCGAT |
| | CGATCACCAG (SEQ ID NO: 5) | CACCAC (SEQ ID NO: 26) |
| T3 | GTGATCGCGTCCTTACCGTT | GTGATCGCGTCCTTACCGTT |
| | TTTTCGGTAAGGACGCGATC | TTTTCGGTAAGGACGCGATC |
| | ACCAG (SEQ ID NO: 6) | ACCAC (SEQ ID NO: 27) |
| T4 | TGATCGCGTCCTTACCGGTT | TGATCGCGTCCTTACCGGTTTT |
| | TTTTCCGGTAAGGACGCGAT | TTCCGGTAAGGACGCGATCAC |
| | CACCAG (SEQ ID NO: 7) | CAC (SEQ ID NO: 28) |
| T4Scr | TACGGTTCCGGCGTTACCTT | TACGGTTCCGGCGTTACCTTTT |
| | TTTTGGTAACGCCGGAACCG | TTGGTAACGCCGGAACCGTAC |
| | TACCAG (SEQ ID NO: 8) | CAC (SEQ ID NO: 29) |
| T5 | GATCGCGTCCTTACCGGTTT | GATCGCGTCCTTACCGGTTTTT |
| | TTTTACCGGTAAGGACGCGA | TTACCGGTAAGGACGCGATCA |
| | TCACCAG (SEQ ID NO: 9) | CCAC (SEQ ID NO: 30) |
| T6 | ATCGCGTCCTTACCGGTTTT | ATCGCGTCCTTACCGGTTTTTT |
| | TTTTAACCGGTAAGGACGCG | TTAACCGGTAAGGACGCGATC |
| | ATCACCAG (SEQ ID NO: 10) | ACCAC (SEQ ID NO: 31) |
| T7 | TCGCGTCCTTACCGGTTCTTT | TCGCGTCCTTACCGGTTCTTTTT |
| | TTTGAACCGGTAAGGACGCG | TGAACCGGTAAGGACGCGATC |
| | ATCACCAG (SEQ ID NO: 11) | ACCAC (SEQ ID NO: 32) |
| | CGCGTCCTTACCGGTTCCTT | |
| T8 | TTTTGGAACCGGTAAGGACG | |
| | CGATCACCAG (SEQ ID | |
| | NO: 12) | |
| T9 | GCGTCCTTACCGGTTCCGTT | |
| | TTTTCGGAACCGGTAAGGAC | |
| | GCGATCACCAG (SEQ ID | |
| | NO: 13) | |
| Forward Linear | CCGGTAAGGACGCGATCAC | CCGGTAAGGACGCGATCACCA |
| (stern + toehold) | CAG (SEQ ID NO: 14) | C (SEQ ID NO: 33) |
| Reverse (Linear) | CAGCAGGGCTCTTCGTCAGC | CAGCAGGGCTCTTCGTCAGCTC |
| | TC (SEQ ID NO: 15) | (SEQ ID NO: 34) |
| Hydrolysis Probe | 5′FAM/TGTTGTCCCATTTCGT | 5′FAM/TGTTGTCCCATTTCGTC |
| | CGGGGTGTTCGTCC 3′Iowa | GGGGTGTTCGTCC 3′Iowa Black |
| | Black (SEQ ID NO: 16) | (SEQ ID NO: 35) |
| | RpoB WT Toehold Hairpin | RpoB Q513L SNP Toehold |
| | Primers | Hairpin Primers |
| T0 | TGGCTCAGCTGGCTGGTGCT | TGGCTCAGCTGGCTGGTGCTTT |
| | TTTTTGCACCAGCCAGCTGA | TTTGCACCAGCCAGCTGAGCCT |
| | GCCA (SEQ ID NO: 17) | (SEQ ID NO: 36) |
| T3 | CTCAGCTGGCTGGTGCTTTT | CTCAGCTGGCTGGTGCTTTTTT |
| | TTGCACCAGCCAGCTGAGCC | GCACCAGCCAGCTGAGCCT |
| | A (SEQ ID NO: 18) | (SEQ ID NO: 37) |
| T4 | TCAGCTGGCTGGTGCCTTTT | TCAGCTGGCTGGTGCCTTTTTT |
| | TTGGCACCAGCCAGCTGAGC | GGCACCAGCCAGCTGAGCCT |
| | CA (SEQ ID NO: 19) | (SEQ ID NO: 38) |
| T4Scr | CGGTGGCCGCTATCGTTTTT | CGGTGGCCGCTATCGTTTTTTT |
| | TTACGATAGCGGCCACCGGC | ACGATAGCGGCCACCGGCCT |
| | CA (SEQ ID NO: 20) | (SEQ ID NO: 39) |
| T5 | CAGCTGGCTGGTGCCGTTTT | CAGCTGGCTGGTGCCGTTTTTT |
| | TTCGGCACCAGCCAGCTGAG | CGGCACCAGCCAGCTGAGCCT |
| | CCA (SEQ ID NO: 21) | (SEQ ID NO: 40) |
| T6 | AGCTGGCTGGTGCCGATTTT | AGCTGGCTGGTGCCGATTTTT |
| | TTTCGGCACCAGCCAGCTGA | TCGGCACAGCCAGCTGAGCC |
| | GCCA (SEQ ID NO: 22) | T (SEQ ID NO: 41) |
| Forward Linear | GGCACCACCAGCTGAGCC | GGCACCAGCCAGCTGAGCCT |
| (stern + toehold) | A (SEQ ID NO: 23) | (SEQ ID NO: 42) |
| Reverse (Linear) | GCCCGGCACGCTCACGTGAC | GC CGGCACGCTCACGTGACA |
| | AG (SEQ ID NO: 24) | G (SEQ ID NO: 43) |
| Hydrolysis Probe | 5′FAM | 5′FAM CCGACTGTTGGCGCTGG |
| | CCGACTGTTGGCGCTGG | 3′Iowa Black (SEQ ID NO: 44) |
| | Iowa Black (SEQ ID NO: 25) | |

Table II. Sequences are provided for the primers detailed in the studies, including common reverse primers, linear control primers, and filled toehold and scrambled stem negative control primers. Fluorescent hydrolysis probes used to detect template-specific amplification products in real-time assays are also shown.

### Example 3

### Optimization of End-Point PCR with Toehold Hairpin Primers

Because it was unclear whether and how the THPs would work in qPCR as well as what background and side reactions they might produce, their ability to generate PCR products of the correct size was first evaluated. PCR conditions were initially optimized as described in Example 1. The THPs were predicted to have melting temperatures of 62.5° C. for KatG and 69.8° C. for RpoB (calculated based on a 2.5 mM MgCl₂ concentration and assuming complete strand displacement). The common second primers for the PCRs were therefore designed to have Tₘ values of 62.9° C. and 73.2° C., respectively. Thermal cycles were designed around these predicted melting temperatures.

The linear positive controls for these assays were primers that had previously yielded efficient amplification and allele discrimination, and that contained the same target-binding sequence as the THP (Table II) but without a competing complement. As negative controls amplifications were performed in the absence of target as well as amplifications with a primer that contained a complementary sequence extension that completely covered the toehold (i.e. a T0 primer) (Table II).

Reactions were assessed by gel electrophoresis to ensure that an amplicon of the correct size was being produced. Initial experiments revealed no difference between T6 and T9 primers. Different conditions were considered that would yield efficient amplification by either T3 or T6 primers yet no amplification in the absence of template or with a T0 primer. Several different buffer conditions and both three-step and two-step PCR cycles were evaluated.

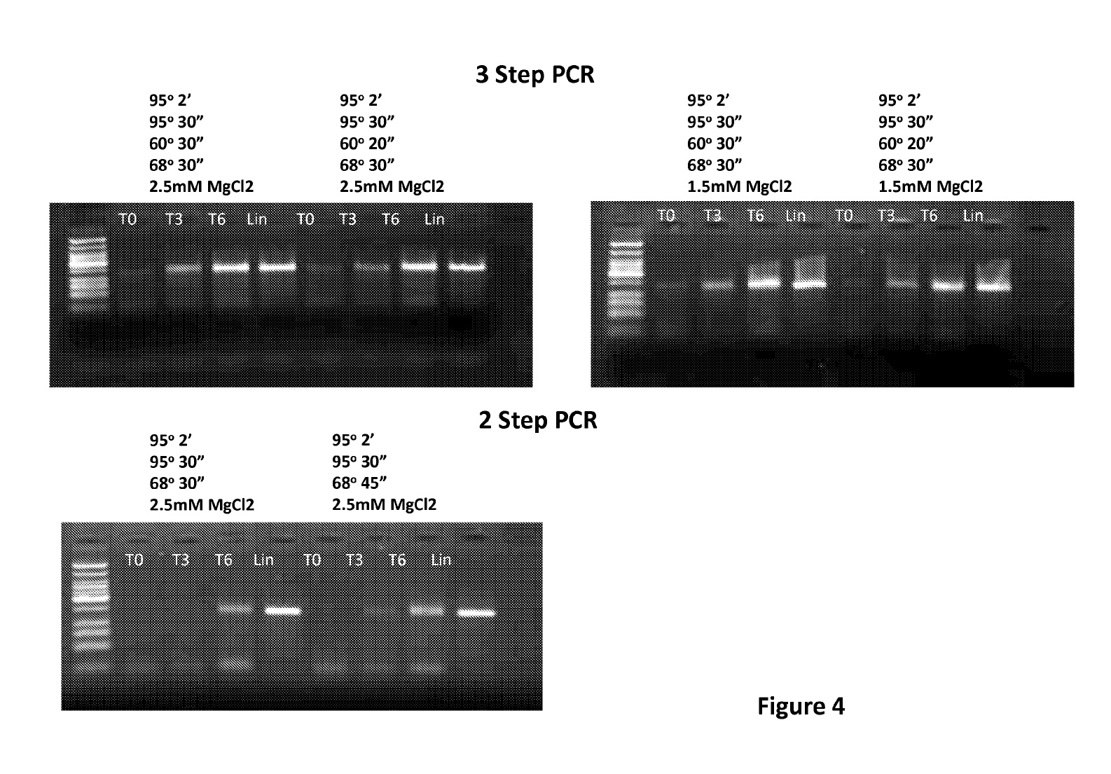

A simple protocol that produced visible bands for the T6 primer and no bands with the T0 primer at 20 cycles with 1 ng of template was a two-step PCR with a 2 min denaturing step at 95° C., and 20 cycles with a 30 s 95° C. denaturing step followed by a 30 s annealing/extension incubation at 68° C. (FIG. 4). These conditions were also amenable to real-time PCR and were therefore used in all further analyses.

### Example 4

### Optimization of Real-Time PCR with Toehold Hairpin Primers

Having shown that THPs could produce bands of the correct size, primer designs and reaction conditions were then further optimized in a real-time PCR assay. It certain cases, shorter toeholds might produce greater discrimination between alleles. However, since the T3 primer gave weak or no bands in end-point PCR, toehold lengths of 4, 5, and 6 nt were tested for amplification and SNP discrimination. Assays were performed using two-step, real-time PCR and conditions similar to those described above but with the inclusion of a 10 min 95° C. incubation to activate the real-time Tag “HotStart” polymerase. To ensure reproducibility and translation to clinical use, a commercial master mix (Fast Universal Probe Master, Rox), a qPCR machine designed for clinical applications (LightCycler 96), and FAM-labeled hydrolysis probes were used. ΔCq, the difference in Cq (i.e. the number of cycles required to achieve a basal signal above background), was designated as a measure of how well the primers discriminate between alleles.

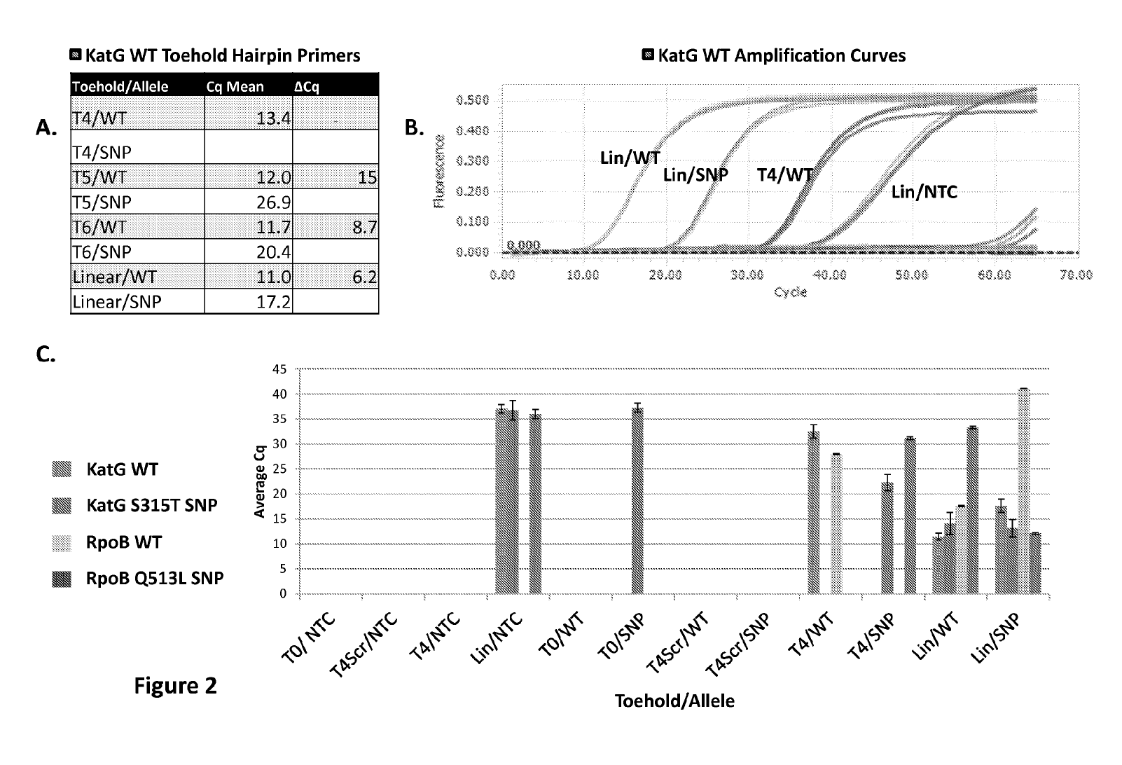

The linear primers (Lin) demonstrated relatively small Cq differences between matched and mismatched targets (ΔCq=6.2). The THPs showed greater discrimination: the T6 primer gave a ΔCq of 8.7, the T5 primer gave a ΔCq of 15, while the T4 primer did not amplify the mismatched target (FIG. 2a). The T4 primers reproducibly gave an average Cq of 32.5 for the wild-type template and showed no amplification through 45 cycles with the mutant template (FIG. 2b, c). These results were in general concordance with the notion that mismatch discrimination by THPs was highly dependent upon the initial contact of the toehold with the template. It should also be noted that these results show much greater ΔCq values than previously published hairpin primers without allele-specific toeholds (Hazbon & Alland, 2004). For example, a hairpin primer with the toehold in the loop of the hairpin yielded a maximum difference in cycle number between a matched template and a single mismatch of 11.2 cycles, as opposed to the 15 or greater cycle differences that we routinely observe.

In order to demonstrate that both strand extension and strand displacement were important for the function of THPs, a primer was generated that was similar to T4, but in which the complementary sequence beyond the toehold was scrambled (T4Scr). The T4Scr primer showed no amplification of either the wild-type or mutant targets.

Having shown that wild-type THPs could discriminate against mutant alleles, it was addressed whether primers specific for the mutant could be readily generated and would in turn similarly discriminate against the wild-type gene. To this end, the 3′ nucleotide on the KatG wild-type (WT) T4 primer was modified from a C to a G (KatG S315T T4). The linear version of the primer gave a ΔCq between mutant and wild-type templates of 9 cycles, while the KatG S315T T4 primer once again did not yield amplification of the mismatched (in this case wild-type) template (FIG. 2c).

### Example 5

### Generalization to Other Genes

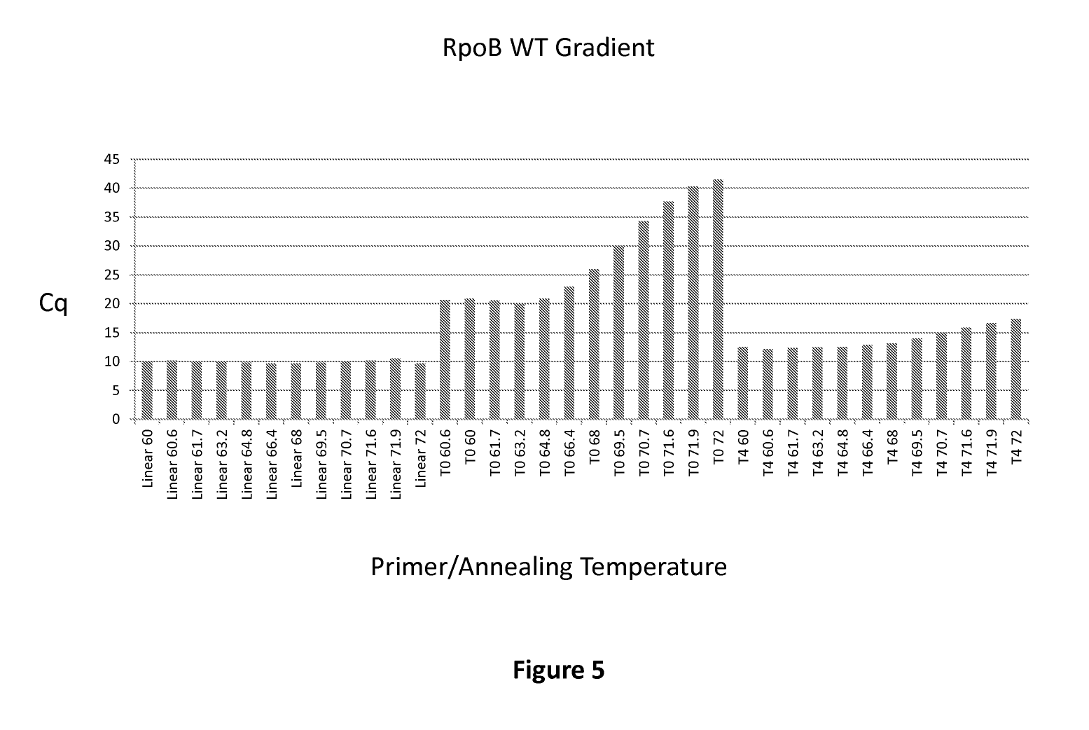

A similar THP design was tested with the RpoB WT gene and its Q513L allele. The previous results with the KatG gene indicated that a T4 primer yielded exquisite discrimination. Therefore primers for RpoB were designed that had only a 4 nt toehold and a 19 bp stem-obscured sequence complementary to the RpoB WT target (Table II). Gradient PCR analysis revealed that the T4 primer performed well in a two-step PCR, with annealing and extension at 72° C. (FIG. 5). Allele discrimination was then verified with 1 ng of template. The linear primer amplified the wild-type allele at a Cq of 17.6 and the Q513L SNP template at a Cq of 41.1, while the RpoB WT T4 primer amplified the wild-type template at an average Cq of 28, but showed no amplification of the mismatched SNP target, even through 60 cycles (FIG. 2c).

The 3′ nucleotide on these primers was changed to be specific for the Q513L SNP (Table II). The resultant linear primer amplified the Q513L template at a Cq of 12.1 and the wild-type at a Cq of 33.3 while, again, the RpoB Q513L T4 primer amplified the Q513L template with an average Cq of 31.2 but showed no amplification of the wild-type, even through 60 cycles (FIG. 2c). This ‘digital discrimination’ of different alleles is useful for diagnostics.

### Example 6

### Efficiencies and Limits of Detection for Toehold Hairpin Primers

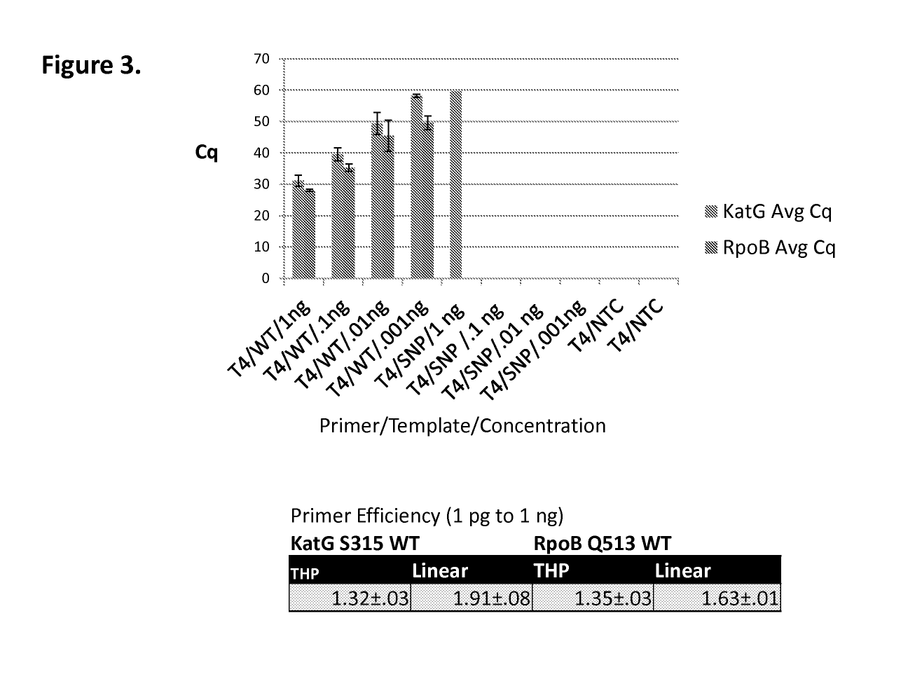

While the THPs showed excellent discrimination between alleles, they were less efficient than their linear counterparts. In some cases, this could limit their applicability for the detection of small amounts of template. Real-time PCR assays were performed with the KatG and RpoB THPs at different template concentrations (between 1 ng and 1 pg) to better establish their efficiencies and limits of detection. Perfectly optimized real-time PCR primers should exhibit an efficiency of 2, indicating a doubling of the target sequence at each cycle. KatG linear primer efficiencies averaged 1.9, while comparable T4 primer efficiencies were 1.3. Efficiencies for the RpoB linear primers averaged 1.6, while the THPs averaged 1.4. Even so, the THPs could detect down to 1 pg of plasmid template relative to no template controls (FIG. 3). In some cases it may be that even smaller amounts of template would not be amplified by THPs, but this could be readily overcome by using nested PCR amplification with linear primers specific for extensions embedded within the THPs.

While THPs are not as efficient as linear primers, they are far more efficient than previously described hairpin primers. The T0 primer specific for the KatG S315T SNP did not show amplification until an average of 37.3 cycles while the T4 primer for the same SNP had an average Cq value of 22.2. This result is very consistent with an exemplary mechanism for toehold binding followed by both extension via Tag polymerase and strand displacement.

### Example 7

### Significance of Certain Embodiments

In summary, a simple primer design method adapted from the field of DNA computation allows synthetic DNA oligonucleotides (or other types of nucleic acid or complementary chemistry, including RNA, PNA, LNA, and so forth) to be generated that can yield exquisitely high discrimination between even single nucleotide mismatches during real-time PCR. The results indicate that mismatch discrimination by toehold hairpin primers was highly dependent upon the initial contact of the toehold with the template, and that the stability of this contact determined whether strand displacement and extension by the polymerase could subsequently occur. Toehold hairpin primers show much greater ΔCq values for SNPs than previously published linear primers. The differentiation between mismatches is typically on the order of 10,000-fold. While more qPCR cycles must be carried out, the diminution in the efficiency of detection is likely to be minimal, especially because of the exquisitely low background amplification exhibited by Toehold Hairpin Primers.

### Example 8

### Determination of the Presence or Absence of a SNP Associated with a Medical Condition

In aspects of the disclosure, there is an individual in need of determination or confirmation of a medical condition in the individual. The individual may or may not have had other tests to determine if the medical condition is present. The individual may or may not have one or more symptoms associated with the medical condition. The individual may already have been treated for the medical condition and the condition needs to be confirmed, or the individual may have been treated for another medical condition, and the condition needs to be determined. The individual may be at risk for having the medical condition, and the chance of the risk is determined. For example, an individual may have a family history of the condition and the SNP is assayed for to determine of the individual is at risk for the condition. Other risk factors include other genetic markers, environmental factors, and so forth.

In some cases, the individual needs to be treated for a diagnosed medical condition, and it needs to be determined whether or not the therapy will be effective in the individual. Part of the diagnosis of the medical condition leading to the determination whether the therapy for it will be effective may or may not include SNP determination, including by methods of the invention.

The individual provides a sample suitable to include cells that have nucleic acid that would allow detection whether or not a SNP was present in the nucleic acid. The sample may be processed prior to the onset of method steps of the invention, such as routine processes to remove cellular debris, proteins, RNA, and so forth, for example. The nucleic acid may be comprised in a tube for analysis or may be present on a microarray, for example. In certain cases, the analysis may be performed on paper, such as FTA® (fast technology for analysis of nucleic acids) paper, including Whatman@ FTA® paper.

A primer as described herein is provided to the nucleic acid sample. The sequence of the primer is dictated by the particular nucleotide or nucleic acid sequence needed to be assayed for in the sample of the particular individual. The primer may be designed such that a wildtype sequence may be identified or confirmed or that a mutation or SNP presence is identified or confirmed. In cases where a SNP is suspected of being present, the primer may be designed such that if a SNP is present, there is a mismatch between the SNP and the primer at that nucleotide, and no PCR amplification would occur on the presence of a suitable polymerase. For example, if the SNP being assayed for is a T at a particular position in the individual's nucleic acid, the primer may have a corresponding T, G, or C at that position, but not an A. An A-containing primer could also be used to obtain a positive signal. If the T is in fact present in the sample, no product would be produced if the primer had a corresponding T, G, or C at that position. Similarly, if a wild-type nucleotide at that position was a T, then a primer having a corresponding T, G, or C at that position would not produce an amplification product.

### Example 9

### Nucleic Acid Capture

In embodiments of the disclosure, toehold hairpin primers as contemplated herein are utilized to capture nucleic acid molecules of interest. In specific embodiments, the toehold hairpin primers are affixed to a substrate, and the substrate/primer entities are exposed to a plurality of nucleic acid molecules of which a fraction of the plurality of molecules is desired to be captured. The capture of the desired nucleic acid molecule(s) occurs upon binding of the toehold hairpin primer to the corresponding nucleic acid molecule, following which the primers are able to extend (or not) depending upon whether or not there is a mismatch. In particular embodiments, the plurality of molecules comprises mRNA from one or more cells. In certain embodiments, the toehold hairpin primers are affixed to a substrate such as a bead, and such as through conjugation.

Toehold hairpin primers conjugated to a solid surface capture target RNA molecules in a specific and efficient manner. 20 uM THP and linear primers with 5′ amine modifications were coupled to 1 micron magnetic beads with —COOH surface modifications (Bangs Laboratories). Conjugated beads were used under various conditions to capture specific RNAs from PBS containing either unprocessed whole Hela or A431 cells or total RNA purified from these cells (Ambion, RNAqueous Kit). Captured RNA was then subjected to gene specific Reverse Transcription (RT) (Roche Transcriptor Reverse Transcriptase) using linear reverse RT primers. PCR or qPCR followed. If capture was performed with THP, a THP primer was used in PCR. For linear capture products, a linear PCR primer was used.

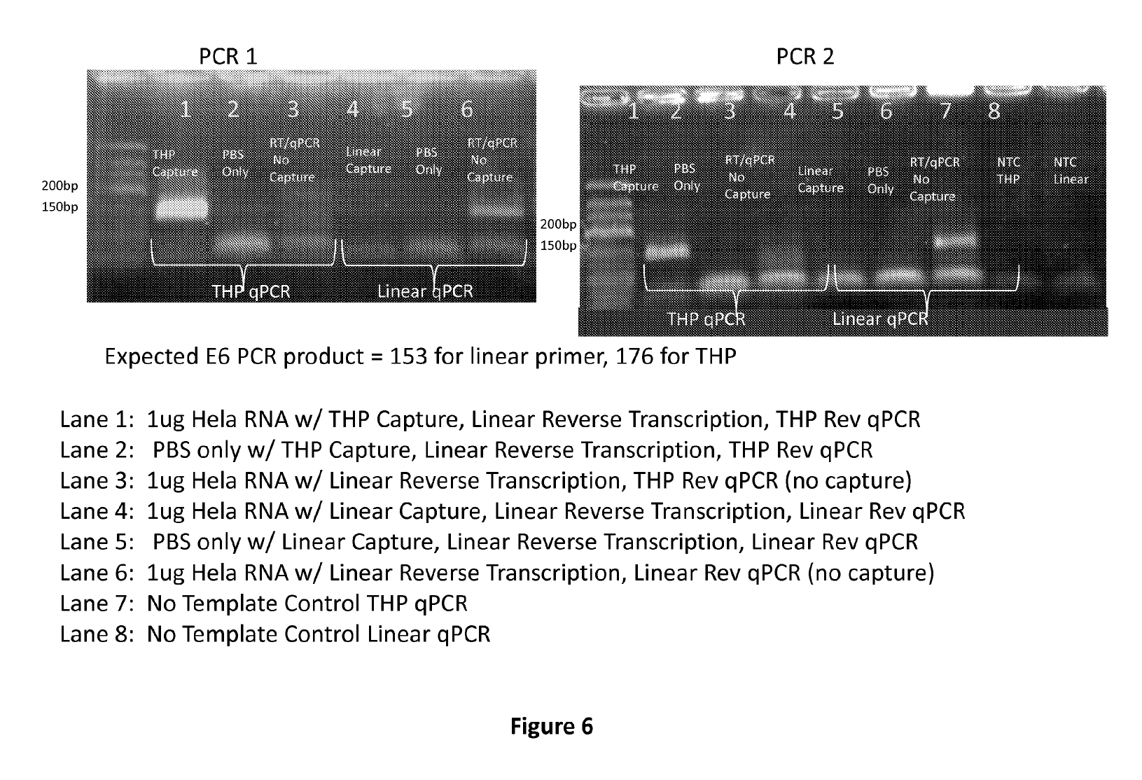

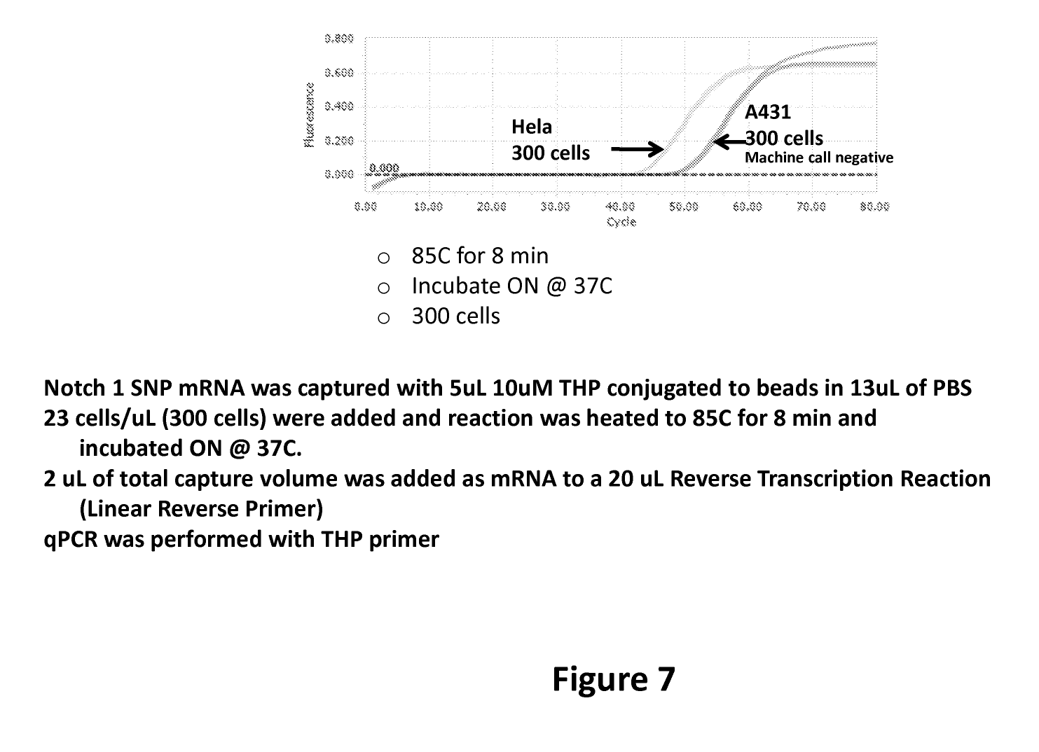

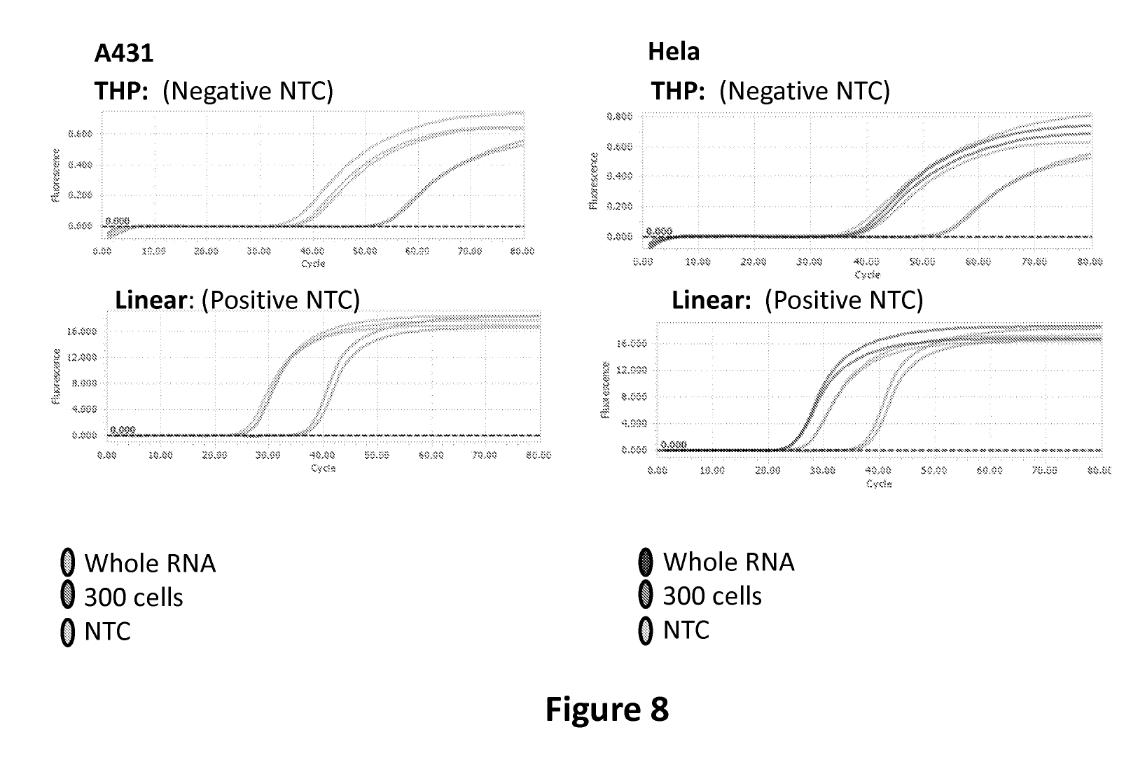

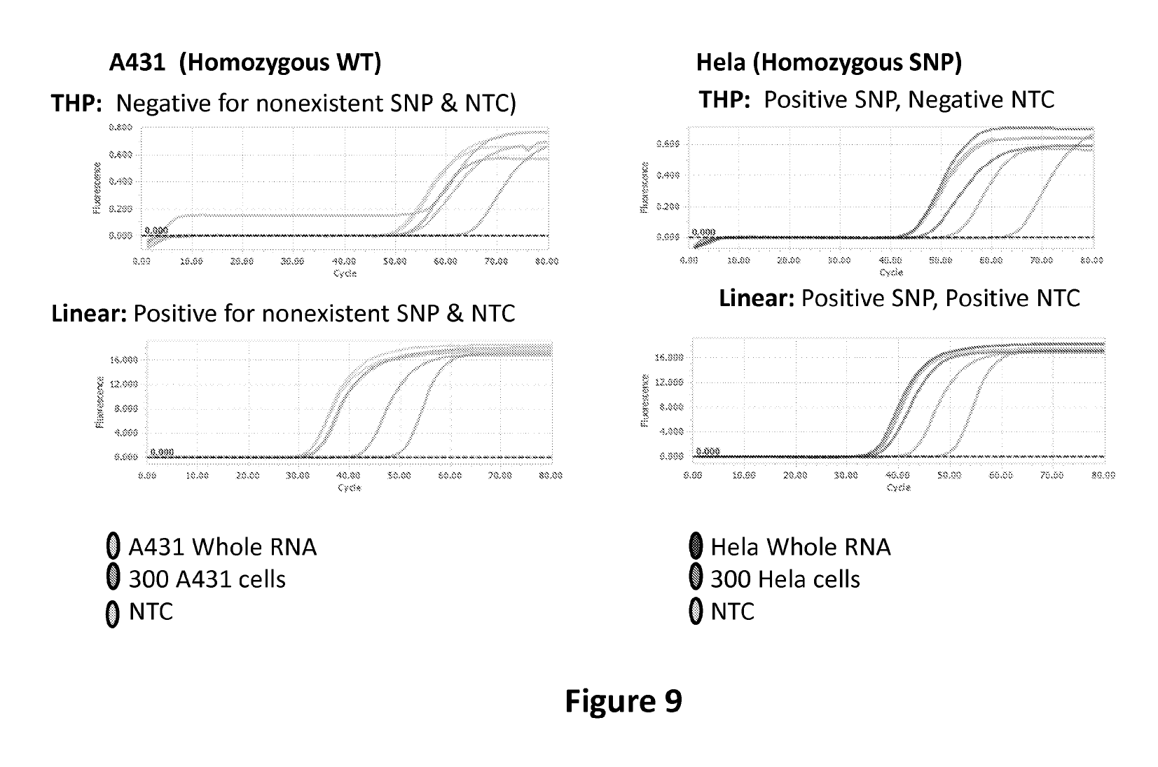

In FIG. 6, THPs specific for the E6 Human Papilloma Virus mRNA expressed in Hela cells demonstrate dramatic enrichment of product from 1 ug total Hela cell RNA. Results shown in FIG. 7 show specificity and sensitivity of THPs conjugated to beads in capturing the downregulated Notch 1 mRNA transcript with a one base pair SNP in a total of only 300 cells subjected only to heat lysis. Homozygous WT A431 cells were used as negative controls. It should be noted that Notch 1 is not downregulated in A431 cells, i.e., there are many times more SNP negative transcripts in A431 sampes than SNP positive transcripts in Hela cells, providing an increased stringency in the experiment. FIG. 8 is a quantitative control for FIG. 7, with 18s rRNA targeted by THPs and linear primers to demonstrate that the same number of A431 and Hela cells (and hence, RNA) were used in the experiment. Note that using the linear primer for capture and qPCR yields positive machine calls for No Template Controls, while using THPs demonstrated negative No Template Controls. FIG. 9 is a second experiment capturing the Notch1 SNP transcript in both whole cells subjected to heat lysis and total RNA purified from A431 and Hela cells.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
Ayyadevara, et al., Anal Biochem. 284:11-18, 2000.
Benenson, Genetics. 13:455-468, 2012.
Chen & Ellington, Curr Opinion Biotechnol. 21:392-400, 2010.
Fan, et al., Nat Rev Genet. 7:632-644, 2006.
Fan, et al., J Clin Microbiol. 41:993-997, 2003.
Farooqi, et al., J Pakistan Med Assoc. 62:767-772, 2012.
Flegal, Vox Sanguinis. 78:109-115, 2000.
Hazbon & Alland, J Clin Microbiol. 42:1236-1242, 2004.
Heym, et al., J Bacteriol. 175:4255-4259, 1993.
Huang, et al., Nucleic Acids Res. 20: 4567-4573, 1992.
Imperiale, et al., Int J Tuber Lung Dis. 17:1088-1093, 2013.
Jurinke, et al., Mol Biotechnol. 26: 147-163, 2004.
Kiepiela, et al., Tuber Lung Dis. 80: 47-56, 2000.
Kim & Misra, Ann Rev Biomed Engineer. 9: 289-320, 2007.
Kostrikis, et al., Science, 279: 1228-1229, 1998.
Kwok, Ann Rev Genomics Human Genet. 2: 235-258, 2001.
Latorra, et al., Human Mutation. 22: 79-85, 2003.
Livak, Genet Anal. 14: 143-149, 1999.
Lyon, et al., Genet Med. 14: 990-1000, 2012.
McGuigan & Ralston, Psychiatric Genetics. 12: 133-136, 2002.
Newton, et al., Nucleic Acids Res. 17: 2503-2516, 1989.
Opel, et al., J Forensic Sci. 55: 25-33, 2010.
Srinivas, et al., Nucleic Acids Res. 41: 10641-10658, 2013.
Syvanen, Nat Rev Genet. 2: 930-942, 2001.
Whitcombe, et al., Nat Biotech. 17: 804-807, 1999.
Williams, J Mol Diagnostics. 3: 98-104, 2001.
Yin, et al., Nature. 451: 318-322, 2008.
Zaczek, et al., BMC Microbiol. 9: 10, 2009.
Zhang & Winfree, J Am Chem Soc. 131: 17303-17314, 2009.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A composition comprising a single stranded primer, said primer comprising a 5′ end, a region of intramolecular complementarity, and a single stranded 3′ end, wherein the single stranded 3′ end comprises at least one designed mismatched nucleotide in relation to a corresponding region of a nucleic acid to which it is complementary.

2. The composition of claim 1, wherein the single stranded 3′ end is between 3 and 15 nucleotides in length.

3. The composition of claim 1, wherein the primer is at least 18 nucleotides in length.

4. The composition of claim 1, wherein the primer is between 18 and 60 nucleotides in length.

5. The composition of claim 1, wherein the primer has a G/C percentage of 40% to 70%.

6. The composition of claim 1, wherein the region of intramolecular complementarity is at least 5 nucleotides in length.

7. (canceled)

8. The composition of claim 1, further comprising a single stranded loop sequence.

9. The composition of claim 8, wherein the single stranded loop sequence is at least 4 nucleotides in length.

10. The composition of claim 8, wherein the single stranded loop sequence is between 4 and 40 nucleotides in length.

11. The composition of claim 8, wherein the loop sequence comprises homopolymeric sequence.

12. The composition of claim 8, wherein the loop sequence comprises random sequence.

13. The composition of claim 8, wherein the loop sequence is specific for a target sequence.

14.-16. (canceled)

17. The composition of claim 1, wherein the designed mismatched nucleotide is present in the primer at the 3′-most nucleotide of the 3′ single stranded end.

18. The composition of claim 1, wherein the designed mismatched nucleotide is present in the primer other than at the 3′-most nucleotide of the 3′ single stranded end.

19.-21. (canceled)

22. The composition of claim 1, wherein the mismatched nucleotide corresponds to a known single nucleotide polymorphism in the nucleic acid.

23. The composition of claim 1, wherein the mismatched nucleotide corresponds to a known wild-type nucleotide in the nucleic acid.

24. A nucleic acid complex, comprising
a primer, said primer comprising a 5′ end, a region of intramolecular complementarity, and a single stranded 3′ end; and
a double stranded nucleic acid having a template strand and a complementarity strand,
wherein said single stranded 3′ end of the primer is complementary to and bound to a region of a corresponding template strand of the double stranded nucleic acid except for one mismatched nucleotide, and wherein the region of complementarity between the primer and template strand is sufficiently short such that upon binding of the primer to the template strand, there is strand displacement of the complementarity strand from the double stranded nucleic acid and there is polymerization from the 3′ end of the primer when in the presence of a polymerase.

25. The complex of claim 24, wherein the region of complementarity between the primer and template strand is between 3 and 15 nucleotides in length.

26.-34. (canceled)

35. The complex of claim 24, wherein the mismatched nucleotide between the primer and the template strand is at the site of a single nucleotide polymorphism.

36. The complex of claim 24, wherein the mismatched nucleotide between the primer and the template strand is at a site suspected of having a single nucleotide polymorphism.

37. The complex of claim 24, wherein the single nucleotide mismatch is present in the complex based on design of the primer.

38. A method of determining the presence or absence of a known nucleotide or known nucleic acid sequence in a sample from an individual, comprising the steps of:
exposing a primer to nucleic acid from the sample, wherein said primer comprises a 5′ end, a region of intramolecular complementarity, and a single stranded 3′ end, wherein the primer binds to nucleic acid from the sample at a region of complementarity between the single stranded 3′ end and the nucleic acid,
wherein when there is a single nucleotide mismatch in the region of complementarity between the single stranded 3′ end of the primer and the nucleic acid, the primer is not able to be polymerized from its 3′ end and no detectable polymerization product is produced, and
wherein when there is not a single nucleotide mismatch in the region of complementarity between the single stranded 3′ end of the primer and the nucleic acid, the primer is able to initiate strand displacement and initiate polymerization from its 3′ end and a detectable polymerization product is produced.

39. The method of claim 38, wherein the primer is designed to include the single nucleotide mismatch in the region of complementarity between the single stranded 3′ end of the primer and the nucleic acid.

40.-54. (canceled)

55. The method of claim 38, wherein when there is a detectable polymerization product produced, the polymerization product is amplified.

56. (canceled)

57. A method of assaying for the presence or absence of a known nucleotide or known nucleic acid sequence in a sample from an individual, comprising the steps of:
assaying for the presence of a polymerization product from a primer bound to a nucleic acid template at a region of complementarity in the template, wherein the region of complementarity comprises the known nucleotide or known nucleic acid sequence in the template and wherein the primer is bound thereto at its single stranded 3′ end, wherein the region of complementarity is no more than 15, 14, 13, 12, 11, 10, 9, 8, 7, 6, 5, or 4 nucleotides in length,
wherein when there is a mismatch in the region of complementarity between the primer and the nucleic acid template, no polymerization product is produced and the presence or absence of the known is determined, or
wherein when there is no mismatch in the region of complementarity between the primer and the nucleic acid template, a polymerization product is produced and the presence or absence of the known is determined.

58. The method of claim 57, wherein the primer is designed to have a single nucleotide mismatch in the region of complementarity.

59. The method of claim 57, wherein the primer is designed to have no mismatches in the region of complementarity.

60. (canceled)

61. A method of capturing one or more desired nucleic acids from a plurality of nucleic acids, comprising the steps of:
exposing a primer-bound substrate to a plurality of nucleic acids,
wherein said primer comprises a 5′ end, a region of intramolecular complementarity, and a single stranded 3′ end, wherein the primer binds to nucleic acid from the sample at a region of complementarity between the single stranded 3′ end and the nucleic acid,
wherein when there is a single nucleotide mismatch in the region of complementarity between the single stranded 3′ end of the primer and the nucleic acid, the primer is not able to be polymerized from its 3′ end and no polymerization product is produced, and
wherein when there is not a single nucleotide mismatch in the region of complementarity between the single stranded 3′ end of the primer and the nucleic acid, the primer is able to initiate strand displacement and initiate polymerization from its 3′ end and a polymerization product is produced; and
subjecting said polymerization product to processing.

62. The method of claim 61, wherein said processing comprises amplification.

63.-68. (canceled)

69. The method of claim 61, wherein the region of complementarity between the single stranded 3′ end of primer and the nucleic acid is no more than 15, 14, 13, 12, 11, 10, 9, 8, 7, 6, 5, or 4 nucleotides in length.
